(19) United States
(12) Patent Application Publication
(10) Pub. No.: US 2017/0100493 A1
(43) Pub. Date: Apr. 13, 2017

(54) RECEPTOR IMAGING SYSTEMS AND RELATED METHODS

(71) Applicant: The General Hospital Corporation, Boston, MA (US)

(72) Inventors: Christin Y. Sander, Boston, MA (US); Joseph B. Mandeville, Melrose, MA (US); Bruce R. Rosen, Lexington, MA (US)

(21) Appl. No.: 15/289,036

(22) Filed: Oct. 7, 2016

Related U.S. Application Data
(60) Provisional application No. 62/238,788, filed on Oct. 8, 2015.

(51) Int. Cl. A61K 49/00; A61K 31/404; A61K 49/06; A61K 51/04; G01R 33/56; A61B 5/055; A61B 6/03; A61B 6/00; G01R 33/48; A61K 31/438; A61B 5/00

(52) U.S. Cl. CPC A61K 49/0004; A61K 31/438; A61K 31/404; A61K 49/06; A61K 51/0446; A61B 5/0042; A61B 5/055; A61B 6/037; A61B 6/501; A61B 6/5247; A61B 5/0035; G01R 33/481; G01R 33/5601; G01R 33/4806

(57) ABSTRACT

A method includes administering to a subject (i) a pharmacological agent that binds to receptors in a subject, and (ii) a radiotracer to alter a functional state and occupancy of the receptors in the subject. The method also includes acquiring imaging data of brain tissue in the subject comprising the receptors. The imaging data include positron emission tomography (PET) imaging data and functional magnetic resonance (fMR) imaging data. The method further includes calculating (i) a dynamic response of the functional state to the pharmacological agent and the radiotracer based on the fMR imaging data, and (ii) a dynamic response of the receptor occupancy to the pharmacological agent and the radiotracer based on the PET imaging data.

### CLAIM OF PRIORITY

This application claims the benefit of U.S. Provisional Application Ser. No. 62/238,788, filed on Oct. 8, 2015. The entire contents of the foregoing are incorporated herein by reference.

### STATEMENT OF GOVERNMENT RIGHTS

This work was supported in part by National Institute of Health grant R90DA023427, P41EB015896, S10RR026666, S10RR022976, S10RR019933 and S10RR017208. The United States government may have certain rights in the invention.

### TECHNICAL FIELD

This specification relates to receptor imaging systems and related methods.

### BACKGROUND

Classifying drugs and their binding properties can generally be determined in vitro. However, in vivo biological systems can be more complex. In some cases, in vitro properties cannot always be directly translated, as they may be modulated by endogenous agents in vivo. Properties such as the efficacy of a drug, affinity, and downstream effects can be modulated in vivo by, e.g., downstream effects and receptor adaptations. Receptor desensitization and internalization (RDI) are synaptic mechanisms that modulate downstream cellular activity in response to G protein-coupled receptor (GPCR) activation by agonist. RDI has been demonstrated in vitro for some GPCR systems, including dopamine D2 receptors (D2R). Measurements in in vitro systems suggest that internalization can occur within minutes of agonist exposure. Receptors may stay internalized for hours or days. Synaptic adaptation mechanisms can affect pharmacodynamics in vivo, and can thereby affect optimal drug doses and strategies to minimize side effects. For example, radiotracers such as spiperone and pimozide (non-benzamides) show binding properties that oppose those predicted from the classical occupancy theory.

### SUMMARY

In one aspect, a method includes administering to a subject (i) a pharmacological agent that binds to receptors in a subject, and (ii) a radiotracer to alter a functional state and occupancy of the receptors in the subject. The method also includes acquiring imaging data of brain tissue in the subject including the receptors. The imaging data include positron emission tomography (PET) imaging data and functional magnetic resonance (fMR) imaging data. The method further includes calculating (i) a dynamic response of the functional state to the pharmacological agent and the radiotracer based on the fMR imaging data, and (ii) a dynamic response of the receptor occupancy to the pharmacological agent and the radiotracer based on the PET imaging data.

In another aspect, one or more computer-readable non-transitory media stores instructions that are executable by a processing device. The instructions, upon execution, cause the processing device to perform operations that include receiving imaging data of a subject representing receptors of the subject after a pharmacological agent and a radiotracer are administered to the subject. The imaging data include PET imaging data and fMR imaging data. The operations further include calculating (i) a dynamic response, to the pharmacological agent and the radiotracer, of a functional state based on the fMR imaging data, and (ii) a dynamic response, to the pharmacological agent and the radiotracer, of a receptor occupancy based on the PET imaging data.

In yet another aspect, a system includes a computing device including a memory configured to store instructions, and a processor to execute the instructions to perform operations. The operations include receiving imaging data of a subject representing receptors of the subject after a pharmacological agent and a radiotracer are administered to the subject. The imaging data include PET imaging data and fMR imaging data. The operations further include calculating (i) a dynamic response, to the pharmacological agent and the radiotracer, of a functional state based on the fMR imaging data, and (ii) a dynamic response, to the pharmacological agent and the radiotracer, of a receptor occupancy based on the PET imaging data.

In some implementations, the pharmacological agent and the radiotracer are administered to the subject substantially simultaneously. In some cases, the pharmacological agent and the radiotracer are administered within 5-10 minutes of each other. In some cases, the pharmacological agent and the radiotracer are administered within 2-3 hours of each other.

In some implementations, the method and/or the operations further include administering an iron oxide contrast agent. The iron oxide contrast agent is administered, for example, before acquiring the imaging data.

In some implementations, the pharmacological agent is administered to the subject parenterally. In some implementations, the radiotracer is administered to the subject parenterally.

In some implementations, the receptor occupancy corresponds to receptor occupancy of the pharmacological agent on the receptors.

In some implementations, the radiotracer is a ligand for the receptors.

In some implementations, acquiring the imaging data includes simultaneously acquiring the PET imaging data and the fMR imaging data. The imaging data is, for example, acquired such that a time interval over which the PET imaging data is acquired overlaps with a time interval over which the fMR imaging data is acquired.

In some implementations, acquiring the imaging data includes sequentially acquiring the PET imaging data and the fMR imaging data. The imaging data is, for example, acquired such that a time interval over which the PET imaging data is acquired does not overlap with a time interval over which the fMR imaging data.

In some implementations, the imaging data include images representing a brain of the subject. The images, for example, represent a region of the brain. The region of the brain includes, for example, one or more of the cerebellum, the putamen, the thalamus, or the cortex.

In some implementations, the dynamic response of the functional state is defined at least in part by a peak value of the functional state. Alternatively or additionally, the dynamic response of the functional state is defined at least in part by a temporal response of the functional state. In some cases, the dynamic response of the functional state is defined at least in part by a distribution of values of the functional state.

In some implementations, the dynamic response of the receptor occupancy is defined at least in part by a peak value of the receptor occupancy. Alternatively or additionally, the dynamic response of the receptor occupancy is defined at least in part by a temporal response of the receptor occupancy. In some cases, the dynamic response of the receptor occupancy is defined at least in part by a distribution of values of the receptor occupancy.

In some implementations, calculating the dynamic response of the functional state includes calculating the dynamic response of the functional state based on a hemodynamic response of the subject. The method and/or the operations further includes, for example, calculating the hemodynamic response based on a cerebral blood volume of the subject measured from the imaging data. The cerebral blood volume is measured, for example, based on the fMR imaging data.

In some implementations, calculating the dynamic response of the receptor occupancy includes calculating the dynamic response of the receptor occupancy based on basal receptor occupancy.

In some implementations, calculating the dynamic response of the receptor occupancy includes calculating the dynamic response of the receptor occupancy based on a binding potential of the receptors.

In some implementations, the method and/or the operations further include quantifying receptor trafficking of the subject. The receptor trafficking is quantified, for example, based on the dynamic response of the functional state. Alternatively or additionally, the receptor trafficking is quantified based on the dynamic response of the receptor occupancy. In some cases, quantifying receptor trafficking includes computing at least one of a desensitization rate constant, an internalization rate constant, a change in affinity of the receptors, or a change in efficacy of the pharmacological agent.

In some implementations, the method and/or the operations further include determining specificity, efficacy, affinity, or neurovascular coupling parameters of the radiotracer. Alternatively or additionally, the method and/or the operations further include determining specificity, efficacy, affinity, or neurovascular coupling parameters of the pharmacological agent.

In some implementations, the method and/or the operations further include classifying the radiotracer based on the dynamic response of the functional state and the dynamic response of the receptor occupancy. Alternatively or additionally, the method and/or the operations further include classifying the pharmacological agent based on the dynamic response of the functional state and the dynamic response of the receptor occupancy. In some cases, classifying the pharmacological agent and/or the radiotracer includes classifying the radiotracer or the pharmacological agent as a classification selected from the group consisting of antagonist, inverse agonist, partial agonist, and full agonist.

In some implementations, the method and/or the operations further include measuring a neurological effect of the pharmacological agent based on the receptor occupancy. Measuring the neurological effect, for example, includes measuring occupancy peak values or response duration after administering the pharmacological agent.

In some implementations, the fMR imaging data is acquired using an fMR imaging device. Alternatively or additionally, the PET imaging data is acquired using an PET imaging device. In some implementations, the system further includes an fMR imaging device to acquire the fMR imaging data representing the receptors. Alternatively or additionally, the system includes an PET imaging device to acquire the PET imaging data representing the receptors.

Advantages of the foregoing may include, but are not limited to, those described below and herein elsewhere. The methods described herein can be used, e.g., to determine the function of a pharmacological agent in vivo to evaluate its functional effects at a given concentration. With simultaneous PET/fMR image acquisition, both occupancy and functional effects of a drug can be determined to predict the potency of the drug in vivo. In this regard, drugs can be classified in a manner that is functionally relevant and that is underpinned by mechanistic understanding of radiotracers and ligands. The methods described herein can overcome limitations of the classical occupancy theory by considering the impact of agonist-induced receptor internalization, which can influence ligand-specific binding rates by altering receptor-ligand affinity. The methods can be used to determine an appropriate dose of a drug to be administered to a subject to have a desired neurological effect.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a representation of imaging data.

FIG. 2 shows a plot of % CBV^{PEAK} versus % Occupancy.

FIG. 3 shows plots of radiotracer activity and % CBV signal versus time.

FIG. 4 shows plots of % CBV versus time for three pharmacological agents.

FIG. 5 shows a schematic of an occupancy and internalization model.

FIG. 6 shows a plot of occupancy and % CBV simulations versus time.

FIG. 7 shows a plot of simulated occupancy versus time.

FIG. 8 shows a plot of expected % CBV responses versus time for different efficacies.

FIG. 9 is a flowchart of an example of a process to calculate dynamic responses to a pharmacological agent and radiotracer.

FIG. 10 is a flowchart another example of a process to calculate dynamic responses to a pharmacological agent and radiotracer.

FIG. 11 is a block diagram of a system that can be used to calculate dynamic responses to a pharmacological agent and radiotracer.

FIG. 12 is a schematic of a computer system.

Like reference numbers and designations in the various drawings indicate like elements.

### DETAILED DESCRIPTION

Measuring RDI and evaluating how its dynamics affect drug action in vivo can be used to optimize therapeutic treatment for neurologic and neuropsychiatric diseases. In the context of drug-receptor interactions, the classical occupancy theory postulates that receptors can be either in a bound or unbound state at the postsynaptic membrane and that binding to or unbinding from receptors causes a functional response. In PET blocking or competition studies, the occupancy model can carry the assumption that changes in radiotracer binding potential directly reflect availability of a given synaptic receptor density. For certain PET ligands, decreases in binding potential can accompany increases in dopamine concentration, as measured by microdialysis. A tight relationship between endogenous dopamine measured from microdialysis and fMR imaging (fMRI) signal changes is also concordant with the classical model. In addition, similar temporal responses from fMRI and PET receptor occupancy due to D2 antagonism support the classical occupancy theory. One consistent observation is that the decrease in binding potential of D2 receptor antagonist radiotracers due to amphetamine lasts much longer than the timecourse of the drug, or the efflux of extracellular dopamine.

The methods and systems described herein can be used to quantify receptor trafficking to classify neuro-receptor function. In particular, receptor desensitization and internalization can be measured to quantify receptor trafficking. The classification can be performed by utilizing a dynamic occupancy model. A temporal response can be elicited by the administration of selected radiotracers and/or candidate molecules. The response can be measured with one or more imaging modalities. The response can be compared with an expected response profile of the tracer and/or candidate molecule. The measurements can be taken through PET/fMR imaging, e.g., simultaneous fMR image acquisition and PET image acquisition, during or after administration of the radiotracers and/or candidate molecules. The measurements can further be used to determine a potency of a drug.

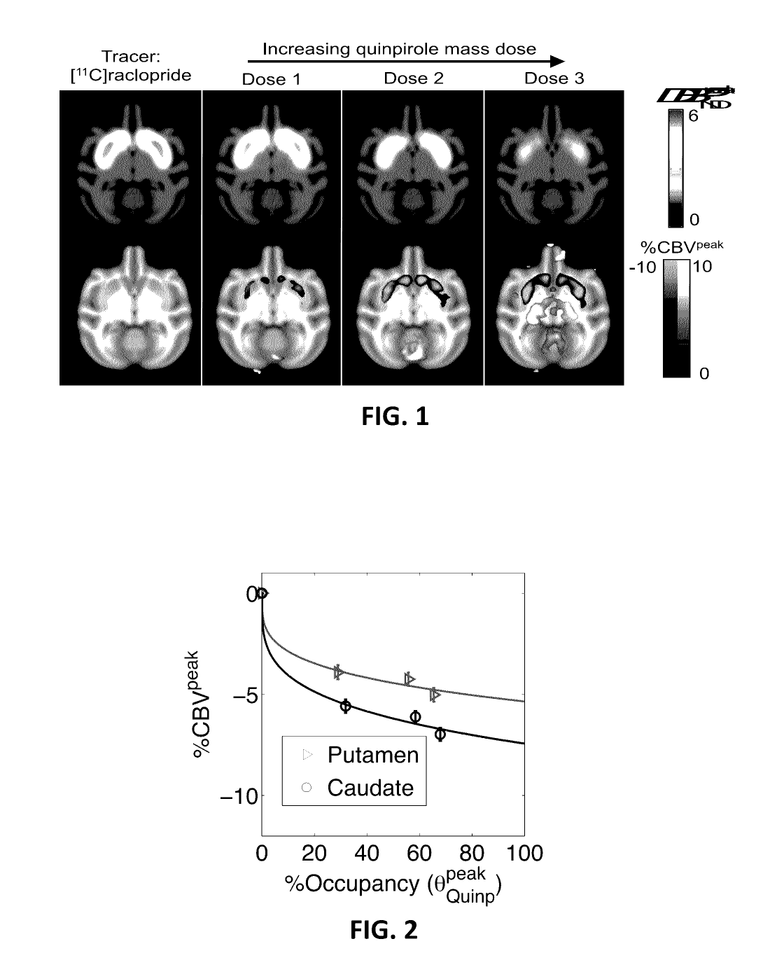

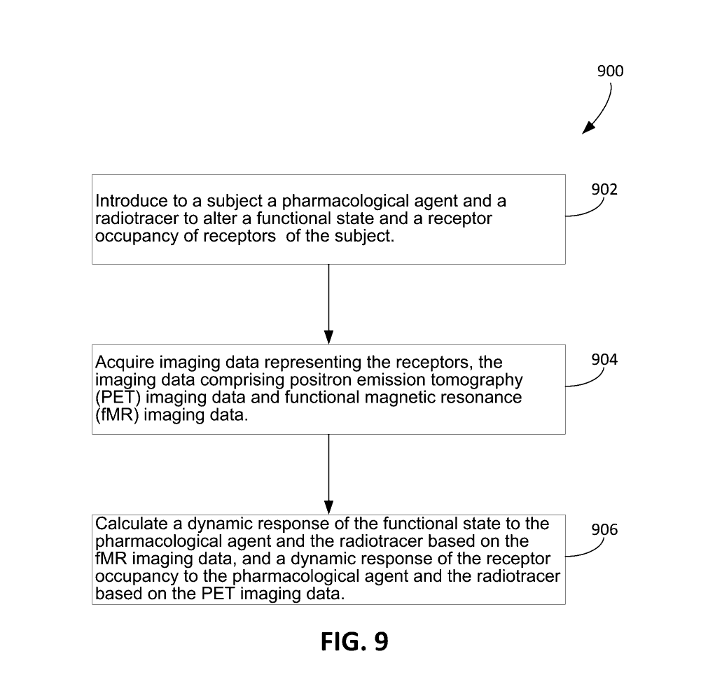

Examples of simulation and experimental results using the systems, methods, and devices presented herein are described with respect to FIGS. 1-8 and in the “Examples” section herein. FIG. 9 depicts a process 900 that can be used to quantify receptor trafficking of a subject. The process 900, for example, can be executed to determine a rate of desensitization associated with a pharmacological agent and/or associated with the subject. The process 900 can also be executed to determine a rate of internalization associated with the pharmacological agent and/or associated with the subject. The process 900 can be used to determine an affinity of receptors of the subject, or a change in efficacy of the pharmacological agent. The efficacy, for example, denotes the strength of a pharmacological response at a given level of receptor occupancy. In some cases, the pharmacological response corresponds to a functional response to pharmacological agent or the radiotracer.

In the example of the process 900, at operation 902, a pharmacological agent and a radiotracer is administered to a subject. The radiotracer is, for example, a ligand for the receptors. In some cases, the pharmacological agent and the radiotracer, when administered, alters a functional state and a receptor occupancy of receptors of the subject. The receptor occupancy corresponds to, for example, a receptor occupancy of the pharmacological agent on the receptors.

At operation 904, imaging data of brain tissue of the subject including the receptors are acquired. The imaging data include, for example, PET imaging data and fMR imaging data.

At operation 906, a dynamic response of the functional state to the pharmacological agent and the radiotracer is calculated based on the imaging data. In addition, a dynamic response of the receptor occupancy to the pharmacological agent and the radiotracer is calculated based on the imaging data. The dynamic response of the functional state is calculated based on, for example, the PET imaging data. The dynamic response of the receptor occupancy is calculated based on, for example, based on the fMR imaging data.

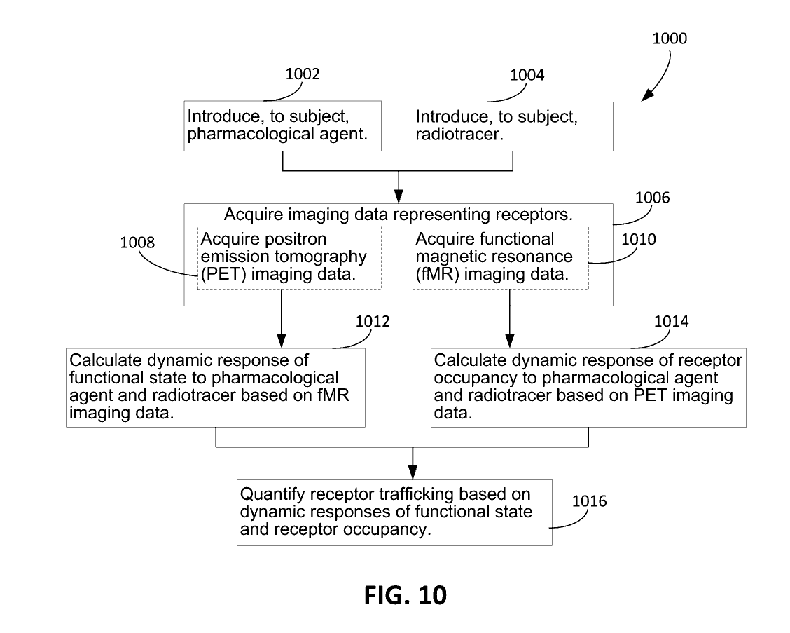

FIG. 10 depicts another example process 1000 that can be used to quantify receptor trafficking of a subject. At operation 1002, a pharmacological agent is administered to the subject.

At operation 1004, a radiotracer is administered to the subject. In some implementations, the operation 902 of the process 900 includes the operations 1002 and 1004.

At operation 1006, imaging data representing receptors of the subject is acquired. In some cases, the operation 1006 further includes operation 1008 and operation 1010. At the operation 1008, PET imaging data are acquired. At the operation 1010, fMR imaging data are acquired. The operation 904 of the process 900, in some cases, includes the operations 1006, 1008, and/or 1010.

At operation 1012, a dynamic response of the functional state of the receptors to the pharmacological agent and the radiotracer is calculated. The dynamic response of the functional state is calculated, for example, based on the fMR imaging data acquired at the operation 1008.

At operation 1014, a dynamic response of the receptor occupancy to the pharmacological agent and the radiotracer is calculated. The dynamic response of the receptor occupancy is calculated, for example, based on the PET imaging data. In some implementations, the operation 906 of the process 900 includes the operation 1012 and/or the operation 1014.

At operation 1016, receptor trafficking is quantified. The receptor trafficking is quantified, for example, based on the dynamic responses of the functional state and the receptor occupancy calculated at the operations 1012 and 1014. In some implementations, the process 900 includes the operation 1016, for example, executed after the operation 906.

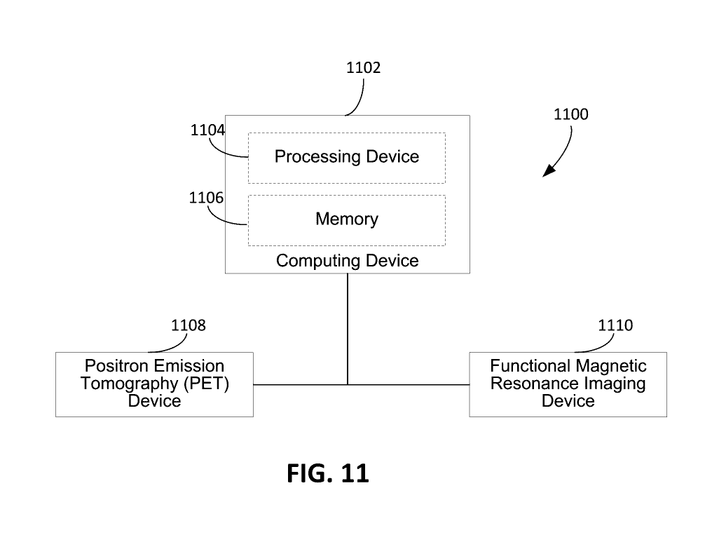

Referring to FIG. 11, a system 1100 includes a computing device 1102 that includes a memory 1104 and a processor 1106. In some implementations, the processor 1106 is configured to execute instructions stored on computer-readable non-transitory media to perform operations, for example, operations associated with the process 900, the process 1000, or other processes disclosed herein. In some cases, the processor 1106 executes instructions stored on the memory 1104 to perform the operations.

The processor 1106, for example, receives imaging data of a subject representing receptors of the subject. The processor 1106 performs an operation to receive the imaging data, for example, after a pharmacological agent and a radiotracer is administered to the subject. In some cases, a human operation administers the pharmacological agent. The imaging data include PET imaging data and fMR imaging data. The processor 1106 performs an operation to calculate a dynamic response of the functional state to the pharmacological agent and the radiotracer based on the fMR imaging data. In addition, the processor 1106 performs an operation to calculate a dynamic response of the receptor occupancy to the pharmacological agent and the radiotracer based on the PET imaging data.

In some implementations, the system 1100 further includes an fMR imaging device 1108 to acquire fMR imaging data representing the receptors of the subject. Alternatively or additionally, the system 1100 includes an PET imaging device 1110 to acquire PET imaging data representing the receptors. The processor 1106, in some cases, operates the fMR imaging device 1108 and/or the PET imaging device 1110 to acquire the imaging data. In some implementations, a scanner system includes both the PET imaging device 1110 and the fMR imaging device 1108. In this regard, in some implementations, in the process 900 and/or the process 1000, the fMR imaging data is acquired using an fMR imaging device, such as the fMR imaging device 1108. Alternatively or additionally, the PET imaging data is acquired using a PET imaging device, such as the PET imaging device 1110.

In some cases, a user interface is placed on the subject to enable the fMR imaging data to be acquired, e.g., to enable the fMR imaging device 1108 to acquire the fMR imaging data. In particular, the user interface is positionable on the head of the patient such that fMR imaging data of the brain can be acquired. In some cases, the user interface includes a polarized transmit coil responsive to magnetic waves generated by the fMR imaging device 1108. Alternatively or additionally, another user interface enables the PET imaging device 1110 to acquire the PET imaging data. The user interface, for example, includes a receive array including 8 or more channels. In some cases, the user interface for fMR image acquisition and the user interface for PET image acquisition are combined into a single wearable unit. The PET imaging device 1110 is, for instance, a BrainPET insert. The fMR imaging device 1108 is, for example, a Tim Trio 3T MR scanner (Siemens AG, Healthcare Sector, Erlangen Germany).

To administer the pharmacological agent and/or the radiotracer, in some cases, the pharmacological agent and/or the radiotracer are injected into the subject. The pharmacological agent and/or the radiotracer are, for example, administered parenterally. The pharmacological agent and/or the radiotracer are, for example, orally administered. In some cases, the pharmacological agent and/or the radiotracer are administered through intravenous administration. The pharmacological agent and/or the radiotracer, in some cases, are administered in the form of a bolus. Alternatively or additionally, the pharmacological agent and/or the radiotracer are administered using a bolus and infusion protocol. In some implementations, the bolus and infusion protocol includes performing an infusion with a k_{bol} value between 40 minutes and 100 minutes, e.g., about 50 minutes, about 80 minutes, etc. In some cases, the pharmacological agent and/or the radiotracer are manually administered, e.g., a human operator manually injects the pharmacological agent and/or the radiotracer. In some implementations, the bolus and infusion protocol includes performing an infusion using a pump, e.g., at a rate of 0.01 mL/second. In some implementations, the pharmacological agent and the radiotracer are administered through a combination of manual injection and computer-implemented operation of a pump. In some implementations, a human operator manually operates the pump to administrate the pharmacological agent and/or the radiotracer.

In some implementations, the pharmacological agent and the radiotracer are administered to the subject substantially simultaneously. The pharmacological agent and the radiotracer are, for example, administered to the subject within 1 to 45 minutes, 1 to 15 minutes, 5 to 10 minutes, 15 to 30 minutes, 30 to 45 minutes, about 30 minutes from one another, etc. In some cases, the pharmacological agent and the radiotracer are administered within 5-10 minutes of one another. In some cases, the pharmacological agent and the radiotracer are administered within 2-3 hours of one another.

In some implementations, the radiotracer and the pharmacological agent are administered at the same time, e.g., in a single injection. In some cases, the pharmacological agent is administered one to several hours before the administration of the radiotracer, e.g., 60 minutes to 4 hours, 60 minutes to 2 hours, about 1 hour, etc. The timing of the pharmacological agent administration relative to the radiotracer administration, for instance, depends on the kinetics of the pharmacological agent and/or the mode of pharmacological agent administration. For example, if administered intravenously, the pharmacological agent can be given either 5 min before, with or up to 60 min after the radiotracer administration. If the drug is given orally or intramuscular, the drug can be given 2-3 hours before or with the radiotracer administration.

In some implementations, an iron oxide contrast agent is administered. The iron oxide contrast agent is, for example, ferumoxytol. The iron oxide contrast agent is administered, e.g., at a concentration of 10 mg/kg of mass of the subject prior to acquiring the fMR imaging data. The iron oxide contrast agent can improve fMR image acquisition detection power. In some implementations, the radiotracer includes an antipsychotic drug.

The radiotracer can include a radioligand that binds to receptors of the subject. The radiotracer is, for example, a non-benzamide, e.g., spiperone, pimozide, etc. In some implementations, the radiotracer includes a dopamine receptor antagonist, e.g., [¹¹C]Raclopride. In some implementations, the radiotracer includes a dopamine D2/D3 receptor antagonist. The dopamine D2/D3 receptor antagonist is, for example, a high affinity dopamine D2/D3 receptor antagonist, e.g., [18F]fallypride. In some implementations, the radiotracer includes a radioligand for PET examination of striatal and neocortical D1-dopamine receptors. The radiotracer is, for example, [11C]NNC112. In some implementations, the radiotracer includes a radioligand for PET examination of extrastriatal D2 dopamine receptors. The radioligand is, for example, [11C]FLB 457.

In some implementations, the pharmacological agent includes a dopamine receptor agonist. The pharmacological agent includes, for example, a high affinity D2/D3 agonist, e.g., quinpirole, which has a K_{D,D2} of 576 nM and a K_{D,D3} of 5 nM. Alternatively or additionally, the pharmacological agent includes a low affinity D2/D3 agonist, e.g., ropinirole, which has a K_{D,D2} of 970 nM and a K_{D,D3} of 61 nM. The dose of the pharmacological agent is, for example, between 0.01 mg/kg and 1 mg/kg, 0.01 and 0.1 mg/kg, 0.1 and 0.3 mg/kg, 0.1 and 1 mg/kg, about 0.1 mg/kg, about 0.2 mg/kg, about mg/kg, etc. The dose of the pharmacological agent is selected such that, for example, the receptor occupancy is within a predefined range, e.g., a predefined range of 30% to 80%. In some implementations, the pharmacological agent is a dopamine receptor antagonist. The pharmacological agent is, for example, a D2 antagonist, e.g., prochlorperazine.

In some implementations, the radiotracer has a relative affinity to internalized receptors of the subject that is defined as the ratio of internal and external affinities. The relative affinity is, for example, greater than or equal to zero. If the relative affinity is zero, the internalized receptors are not accessible to the radiotracer. If the relative affinity is one, the internalized receptors have equal affinity to external receptors. Other values of the relative affinity can indicate that internalized receptors have either decreased or increased affinity.

The fMR imaging data represent, in some cases, maps indicating a binding potential of the pharmacological agent and/or the radiotracer. In some implementations, the fMR imaging data are acquired using parallel acquisitions, e.g., generalized autocalibrating partially parallel acquisitions (GRAPPA). The parallel acquisitions are executed to acquire imaging data in, for example, the anterior-posterior direction. In some implementations, whole-brain fMR imaging data are acquired. The whole-brain fMR imaging data are acquired, for example, using multi-slice echo-planar imaging (EPI). In some examples, motion correction is applied to correct for motion of the subject during acquisition of the fMR imaging data.

In some implementations, the PET imaging data undergo a reconstruction process to generate reconstructed PET imaging data. The reconstruction process includes, for example, execution of a 3D Poisson ordered-subset expectation maximization algorithm using prompt and variance-reduced random coincidence events. In some cases, normalization, scatter, and/or attenuation sinograms are included in the reconstructed PET imaging data. In some examples, motion correction is applied to correct for motion of the subject during acquisition of the PET imaging data.

In some implementations, the PET imaging data and/or the fMR imaging data include imaging data representing a region of a brain of the subject. The PET imaging data and/or the fMR imaging data include, for example, imaging data representing one or more regions of interest, e.g., the cerebellum, the putamen, the thalamus, the cortex, etc., of the brain. The neurological effect of the pharmacological agent and/or the radiotracer can be localized to a specific region. In this regard, in some implementations, the imaging data include representations of the localized region. In some cases, the PET imaging data and the fMR imaging data are acquired substantially simultaneously. The intervals within which the PET imaging data and the fMR imaging data are acquired, for example, overlap by 1 to 120 minutes, e.g., by 1 to 30 minutes, 30 to 60 minutes, 60 to 90 minutes, 90 to 120 minutes, about 20 minutes, about 60 minutes, or about 100 minutes.

In some cases, the PET imaging data and the fMR imaging data are acquired sequentially. In some cases, if acquired sequentially, the PET imaging data and the fMR imaging data are acquired over time intervals that do not overlap. The process 1000 includes, for example, an operation in which the PET imaging data and the fMR imaging data are analyzed to determine a function to relate the time interval of the PET imaging data and the time interval of the fMR imaging data. In some implementations, the PET imaging data and the fMR imaging data are acquired with a delay between the PET imaging data being acquired and the fMR imaging data being acquired. The delay is sufficient to, for example, reduce the influence of prior administrations of pharmacological agents or radiotracers, e.g., 1 to 3 weeks.

In some implementations, the PET imaging data and fMR imaging data are registered to a stereotaxic space, e.g., the Saleem-Logothetis stereotaxic space or MNI human brain template. The imaging data are registered, for example, using an affine transformation. The PET imaging data, e.g., acquired using EPI, are aligned using an affine transformation and local distortion fields. After the imaging data are acquired, a motion-correcting function and/or a smooth function are applied to the fMR imaging data, e.g., using a 2.5 mm Gaussian kernel, using a statistical analysis implementing a general linear model. The dynamic responses are, for example, calculated by applying a gamma-variate function. The time-to-peak for the gamma-variate function is, for example, adjusted to minimize the χ²/degree-of-freedom of the GLM fit to the imaging data.

The pharmacological agent and/or the radiotracer can be considered a ligand that can have specific properties that can be determined using the methods described herein. In some implementations, the dynamic responses are calculated using a dynamic occupancy model, e.g., that considers the pharmacological agent and/or the radiotracer to be a ligand for receptors of the subject. The dynamic occupancy model, for example, includes a neurovascular coupling model that, for example, defines a relationship between receptor occupancy and cerebral blood volume (CBV). The CBV corresponds to, for example, a % CBV of brain volume. The CBV is measured, for instance, based on the fMRI imaging data. 100.

The dynamic response of the functional state of the receptors is calculated, for example, based on a timecourse of the functional state, e.g., the functional state over a period of time, the CBV over a period of time, etc. The dynamic response of the functional state of the receptors, for instance, includes a transient response that can be detected from the timecourse of the CBV.

In some examples, the dynamic response of the functional state is defined by a peak value of the functional state and/or a temporal response of the functional state. The dynamic response is determined, for example, based on a peak value of the functional state and/or a temporal response of the functional state, e.g., a duration between the peak and a baseline of the functional state determined from the timecourse of the functional state. The baseline corresponds to the functional state prior to administering the pharmacological agent and the radiotracer to the subject. In some cases, the process further includes an operation to detect the peak value of the functional state, the time at which the peak value of the functional state occurs, and/or a temporal response of the functional state. A value of the functional state is, for example, determined at the occupancy peak values. In some implementations, the peak value of the functional state is detected within a few minutes after the administration of the pharmacological agent and the radiotracer, e.g., 30 seconds to 5 minutes after the administration of the pharmacological agent and the radiotracer. In some implementations, the process further includes an operation to detect the temporal response of the functional state. The temporal response occurs, for example, over a period of 5 minutes to 35 minutes, e.g., 10 to 30 minutes, about 10 minutes, about 20 minutes, about 30 minutes, etc.

The dynamic response of the functional state is, in some cases, calculated based on a hemodynamic response of the subject. The hemodynamic response is, for example, calculated using the neurovascular coupling model described herein. The hemodynamic response is calculated, for example, based on the CBV of the subject measured from the imaging data, e.g., the fMR imaging data. In some cases, the dynamic response of the functional state is calculated based on an efficacy of the ligand. In some cases, the dynamic response of the functional state is calculated based on a total density of receptors for the subject. In some cases, the dynamic response of the functional state is calculated based on a change in occupancy of the ligand.

In some cases, the dynamic response of the receptor occupancy is defined by a peak value of the receptor occupancy and/or a temporal response of the receptor occupancy. The receptor occupancy is determined, for example, based on a PET time activity curve derived from the PET imaging data. The dynamic response of the receptor occupancy is, for example, determined based on a basal receptor occupancy. The basal receptor occupancy corresponds to, for example, the occupancy prior to administering the pharmacological agent and the radiotracer to the subject. The occupancy is, for instance, a percent receptor occupancy. The dynamic response is determined, for example, based on a peak value of the receptor occupancy and/or a temporal response of the receptor occupancy, e.g., a duration between the peak and the basal occupancy of the receptors. In some cases, the process further includes an operation to detect the peak value of the receptor occupancy, to detect a time at which the peak value of the receptor occupancy occurs, and/or to detect a temporal response of the receptor occupancy. In some implementations, the peak value of the receptor occupancy is detected within a few minutes after the administration of the pharmacological agent and the radiotracer, e.g., 30 seconds to 20 minutes after the administration of the pharmacological agent and the radiotracer. The temporal response occurs, for example, over a period of 5 minutes to 40 minutes, e.g., 10 to 30 minutes, about 10 minutes, about 20 minutes, about 30 minutes, etc. In some implementations, the dynamic response of the functional state is defined by a distribution of values for the functional state.

The dynamic response of the receptor occupancy is calculated, for example, based on a timecourse of the receptor occupancy, e.g., the receptor occupancy over a period of time. In some cases, using the dynamic occupancy model, the dynamic response of the receptor occupancy is calculated based on a binding potential of the ligand. In some implementations, the occupancy of the receptors corresponds to the number of bound receptors divided by the total number of receptors. The receptor occupancy is estimated, for example, as a relative change in the binding potential over a period of time. The binding potential, for example, corresponds to a dynamic binding potential determined based on the PET imaging data. The dynamic binding potential is time-dependent. The binding potential is, for example, determined based on an affinity of the ligand for a target receptor of the subject. In some cases, the binding potential is determined based on an amount of internalization of the receptors of the subject. In some cases, using the dynamic occupancy model, the dynamic response of the receptor occupancy is calculated based on an association rate and/or a dissociation rate of the ligand. Alternatively or additionally, using the dynamic occupancy model, the dynamic response of the receptor occupancy is calculated based on a desensitization rate and/or an internalization rate of the receptors associated with the ligand. In some implementations, the dynamic response of the receptor occupancy is defined by a distribution of values for the receptor occupancy.

In some implementations, a process described herein includes an operation to quantify receptor trafficking of the subject. A quantity representing the receptor trafficking is, for example, calculated based on the dynamic response of the functional state and the dynamic response of the receptor occupancy. In some cases, the quantity representing the receptor trafficking is defined by a desensitization rate constant. The desensitization rate constant is determined, for example, based on the dynamic occupancy model. In some cases, the quantity representing the receptor trafficking is defined by an internalization rate constant. Alternatively or additionally, the receptor trafficking is defined by a receptor desensitization and internalization rate constant, e.g., that accounts for both a rate of desensitization and a rate of internalization of receptors. The internalization rate constant, the desensitization rate constant, and/or the receptor desensitization and internalization rate constant are, in some cases, predefined quantities that are selected based on empirically derived data measuring the occupancy and the functional state associated with a particular ligand. In some cases, the quantity representing the receptor trafficking is defined by a change in affinity of the receptors. In some cases, the quantity representing the receptor trafficking is defined by a change in efficacy of the pharmacological agent.

In some implementations, the receptor trafficking is quantified as a temporal divergence between the dynamic response of the receptor occupancy and the dynamic response of the functional state. The dynamic response of the receptor occupancy includes, for example, an elevated response that lasts longer than a response of the dynamic response of the functional state. In this regard, a PET signal is in some cases elevated for a duration longer than a duration of the elevated response of an fMR signal. A degree of the temporal divergence is, in some cases, indicative of the receptor trafficking, and hence is usable to classify the pharmacological agent and/or the radiotracer, e.g., to classify a desensitization or an internalization associated with the pharmacological agent and/or the radiotracer.

In some implementations, a process described herein includes an operation to determine a specificity, an efficacy, an affinity, or neurovascular coupling parameters of the radiotracer or the pharmacological agent. In some implementations, a process described herein includes an operation to classify the radiotracer or the pharmacological agent. The radiotracer or pharmacological agent is, for example, classified based on the dynamic response of the functional state and the dynamic response of the receptor occupancy. In some cases, the radiotracer or the pharmacological agent is classified as an antagonist, an inverse agonist, a partial agonist, or a full agonist.

The pharmacological agent and/or the radiotracer can be classified using one of the parameters measured or determined herein. The classification is based on, for example, the temporal response, the peak value of the functional state, and/or the time at which the peak value of the functional state occurs. Alternatively or additionally, the classification is based on, for example, the temporal response of the receptor occupancy, the peak value of the receptor occupancy, and/or the time at which the peak value of the receptor occupancy occurs. In some implementations, the classification is further based on a predefined affinity or potency of the pharmacological agent and/or the radiotracer. In some cases, the classification of the pharmacological agent is based on an efficacy of the pharmacological agent, a dosing regimen of the pharmacological agent, an in vivo affinity of the pharmacological agent, an in vivo function at a target due to the pharmacological agent, and/or a target engagement and modulation of a target due to the pharmacological agent.

In some implementations, a process described herein includes an operation to measure a neurological effect on the subject of the pharmacological agent. The neurological effect is measured, for example, based on the receptor occupancy. In some cases, the occupancy peak values, after the pharmacological agent is administered, are measured to determine the neurological effect. Alternatively or additionally, CBV peak values are measured to determine the neurological effect.

In some implementations, a process described herein includes an operation to select a dosing regimen of a pharmacological agent to administer to the subject based on the dynamic responses. The dosing regimen of the pharmacological agent is selected, for example, based on the rate of desensitization or the rate of internalization determined from the dynamic responses. The pharmacological agent is, for instance, used to treat a disorder such as a movement disorder, a psychiatric disease, a neurological disease, substance abuse, etc. The disorder is, for instance, schizophrenia, anxiety, or depression. In some cases, the dosing regimen includes a therapeutic time window and/or an ideal dose for the pharmacological agent. Alternatively or additionally, the dosing regimen is determined based on a target receptor occupancy to be achieved and/or a functional effect on brain signaling to be achieved. If the pharmacological agent includes an antipsychotic, for example, the dosing regimen is selected to achieve a target receptor occupancy, e.g., between 50% and 80% receptor occupancy, and/or a target functional state, e.g., target brain signaling response.

In some cases, baseline levels of neurotransmitters of the subject are different, e.g., elevated or depressed, relative to normal levels. The subject, for example, has a disease that causes the decreased or increased levels of neurotransmitters. In some cases, due to the decreased or increased levels of the neurotransmitters, a rate at which the receptors generate signals is different relative to normal rates. If the disorder is schizophrenia, in some cases, baseline dopamine levels are elevated and receptor function is altered. The methods using the dynamic occupancy model can be used to diagnose the subject with a disorder. The methods can enable a biological quantity, such as the receptor occupancy, the functional state, or a related parameter to be measured. The biological quantity can be used as a biomarker for diagnosis of a disorder. In this regard, in some implementations, based on imaging data acquired using the methods described herein, the subject can be diagnosed with a disorder, e.g., schizophrenia, anxiety, depression, etc. In particular, the biological quantity measured can be compared with a normal value expected of a subject without the disorder, e.g., a human without the disorder. In some cases, the dynamic response of the functional state and/or the dynamic response of the receptor occupancy are compared to expected dynamic responses due to the pharmacological agent and/or the radiotracer.

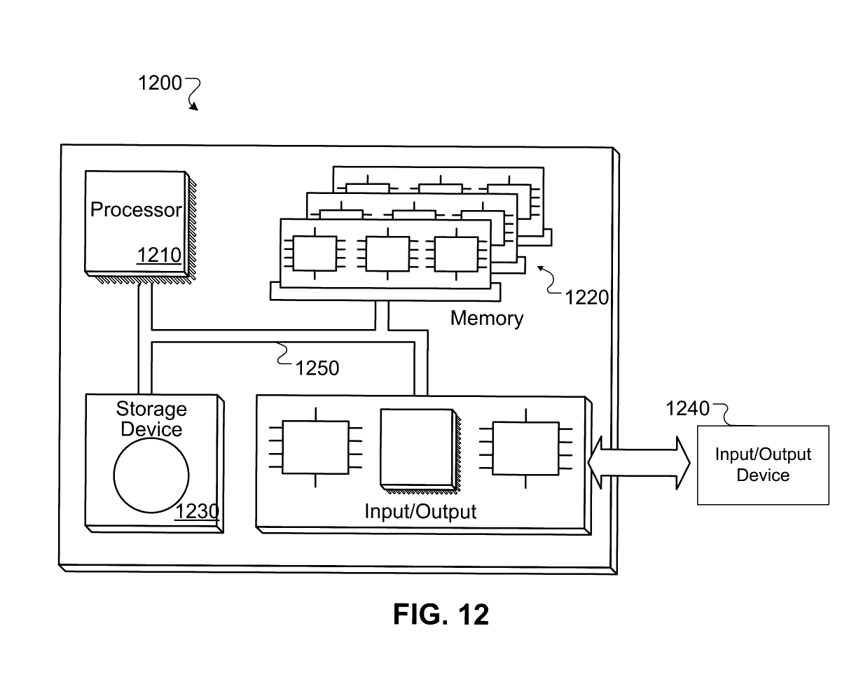

Controllers and any associated components described herein can be part of a computing system that facilitates control of the insertion systems according to processes and methods described herein. FIG. 12 is a schematic diagram of an example of a computer system 1200, e.g., the computing device 1102, that can be used to implement a controller, e.g., including the processor 1106, described in association with any of the computer-implemented methods described herein. The system 1200 includes a processor 1210, a memory 1220, a storage device 1230, and an input/output device 1240. Each of the components 1210, 1220, 1230, and 1240 are interconnected using a system bus 1250. The processor 1210 is capable of processing instructions for execution within the system 1200. In some examples, the processor 1210 is a single-threaded processor, while in some cases, the processor 1210 is a multi-threaded processor. The processor 1210 is capable of processing instructions stored in the memory 1220 or on the storage device 1230 to display graphical information for a user interface on the input/output device 1240.

Memory storage for the system 1200 can include the memory 1220 as well as the storage device 1230. The memory 1220 stores information within the system 1200. The information can be used by the processor 1210 in performing processes and methods described herein. In some examples, the memory 1220 is a computer-readable storage medium. The memory 1220 can include volatile memory and/or non-volatile memory. The storage device 1230 is capable of providing mass storage for the system 1200. In general, the storage device 1230 can include any non-transitory tangible media configured to store computer readable instructions. Optionally, the storage device 1230 is a computer-readable medium. Alternatively, the storage device 1230 may be a floppy disk device, a hard disk device, an optical disk device, or a tape device.

The system 1200 includes the input/output device 1240. The input/output device 1240 provides input/output operations for the system 1200. In some examples, the input/output device 1240 includes a keyboard and/or pointing device. In some cases, the input/output device 1240 includes a display unit for displaying graphical user interfaces.

The features of the methods and systems described in this application can be implemented in digital electronic circuitry, or in computer hardware, firmware, or in combinations of them. The features can be implemented in a computer program product tangibly stored in an information carrier. The information carrier can be, for example, a machine-readable storage device, for execution by a programmable processor. Operations can be performed by a programmable processor executing a program of instructions to perform the functions described herein by operating on input data and generating output. The described features can be implemented in one or more computer programs that are executable on a programmable system including at least one programmable processor coupled to receive data and instructions from, and to transmit data and instructions to, a data storage system, at least one input device, and at least one output device. A computer program includes a set of instructions that can be used, directly or indirectly, in a computer to perform a certain activity or bring about a certain result. A computer program can be written in any form of programming language, including compiled or interpreted languages. The computer program can be deployed in any form, including as a stand-alone program or as a module, component, subroutine, or other unit suitable for use in a computing environment.

Generally, a computer will also include, or be operatively coupled to communicate with, one or more mass storage devices for storing data files. Such devices can include magnetic disks, such as internal hard disks and removable disks, magneto-optical disks, and optical disks. Storage devices suitable for storing the computer program instructions and data include all forms of non-volatile memory, including by way of example semiconductor memory devices, such as EPROM, EEPROM, and flash memory devices, magnetic disks such as internal hard disks and removable disks, magneto-optical disks, and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Alternatively, the computer can have no keyboard, mouse, or monitor attached and can be controlled remotely by another computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network. The relationship of client and server arises by virtue of computer programs running on the respective computers and having a client-server relationship to each other.

The processor 1210 carries out instructions related to a computer program. The processor 1210 can include hardware such as logic gates, adders, multipliers and counters. The processor 1210 can further include a separate arithmetic logic unit (ALU) that performs arithmetic and logical operations.

### EXAMPLES

Implementations are further described in the following non-limiting examples.

### Example 1: Model

Based on the classical occupancy theory, receptor occupancy by agonist can cause a downstream functional response. In some cases, a neurovascular coupling model is used to relate receptor occupancy directly to changes in cerebral blood volume (CBV) as measured by fMRI in the case of an antagonist that displaces the endogenous agonist dopamine. There are other biological mechanisms that can alter predictions made from a classical occupancy model, such as receptor desensitization and internalization.

A functional hemodynamic response can be expressed in terms of changes in receptor occupancies:

$$( Eq . 1 )$$

$$Δ H = {\sum }_{R = 1}^{\# receptors} {\sum }_{L = 1}^{\# ligands} {N}_{R} {ɛ}_{R , L} {B}_{max , R} {Δθ}_{R , L} . \qquad$$

This equation describes changes in function (hemodynamics H) for ligands L binding at receptor R. The parameter N_{R} denotes the neurovascular coupling constant at R, the parameter ε_{R,L} denotes the efficacy of ligand L at R, and the parameter B_{max,R} denotes the total density of receptors R and Δθ_{R,L} the change in occupancy of the ligand at R.

In this context, the efficacy c of an antagonist is zero and that of a full agonist is 1. Partial agonists have an efficacy between 0 and 1. Inverse agonists have an efficacy of −1.

The functional response can be measured by fMRI, e.g., by determining CBV from the fMR imaging data, so that the coupling model describes the fMRI signal model. If a ligand L is specific to D2R (e.g. a D2 agonist as in this work) is administered, the agents contributing to the CBV response the ligand L and endogenous dopamine (DA), which is displaced according to the law of mass action:

ΔCBV(t)=N_{D2}ε_{L}Bₘₐₓθ_{L}(t)−N_{D2}BₘₐₓΔθ_{DA}(t). (Eq. 2)

If a linear approximation between changes in occupancies of DA and drug is assumed, where (Δθ_{DA}=−θ_{DA}⁽⁰⁾θ_{L} (10), then a steady-state model results that is not directly dependent on any affinities or rate constants:

ΔCBV(t)=N_{D2}Bₘₐₓθ_{L}(t)(ε_{L}−θ_{DA}⁽⁰⁾). (Eq. 3)

The PET measurement model describes binding potential BP as a sum over all receptor pools (external and internal) weighted by the respective radiotracer affinity for each pool. The affinity a may be different for external (aₑₓₜ) or internal (aᵢₙₜ) tracer-receptor affinities:

$$BP = \frac{{B}_{ext}}{{K}_{D , ext}} + \frac{{B}_{int}}{{K}_{D , int}} = {a}_{ext} {B}_{ext} + {a}_{int} {B}_{int} .$$

At baseline (t=0), it can be assumed that no receptors are internalized.

With the administration of an internalization-inducing ligand L, receptors may become internalized and affect BP measures.

BP⁽⁰⁾=aₑₓₜ(Bₘₐₓ−B_{DA}⁽⁰⁾)

BP=aₑₓₜ(Bₘₐₓ−B_{DA}−B_{L}−Bᵢₙₜ)+aᵢₙₜBᵢₙₜ.

Receptor occupancy, as measured by PET, (θ_{PET}) is defined as the relative change in BP:

$${θ}_{PET} = \frac{{BP}^{( 0 )} - BP}{{BP}^{( 0 )}}$$

Substituting the expressions for binding potentials (BP⁽⁰⁾) at baseline, and BP after the drug challenge), the expression can be simplified to the following:

$$( Eq . 4 ) \\ {θ}_{PET} = \frac{{θ}_{L} + ( {θ}_{DA} - {θ}_{DA}^{( 0 )} ) + ( 1 - α ) {θ}_{int}}{( 1 - {θ}_{DA}^{( 0 )} )} \qquad$$

The above equation expresses occupancy, measured using PET imaging data, as a sum of receptors bound externally by ligand L and dopamine and internally by ligand L. θ denotes the occupancy (the number of bound receptors B divided by the total number of receptors Bₘₐₓ) for each receptor pool. The relative affinity of the radiotracer to internalized receptors is described by α, which is defined as the ratio of internal and external affinities:

$$α = \frac{{α}_{int}}{{α}_{ext}} .$$

The relative affinity can take values from α≧0. If α=0, internalized receptors are not accessible to the radiotracer and if α=1, internalized receptors have equal affinity to external receptors. Any other value of α means that internalized receptors have either decreased or increased affinity.

The relation of occupancy measured by PET to experimental outcome measures, such as (dynamic) binding potential is then:

$${θ}_{PET} = ( \frac{Δ DBP}{{BP}^{( 0 )}} ) = ( 1 - \frac{DBP}{{BP}^{( 0 )}} ) \\ ∴ DBP = ( 1 - {θ}_{PET} ) {BP}^{( 0 )}$$

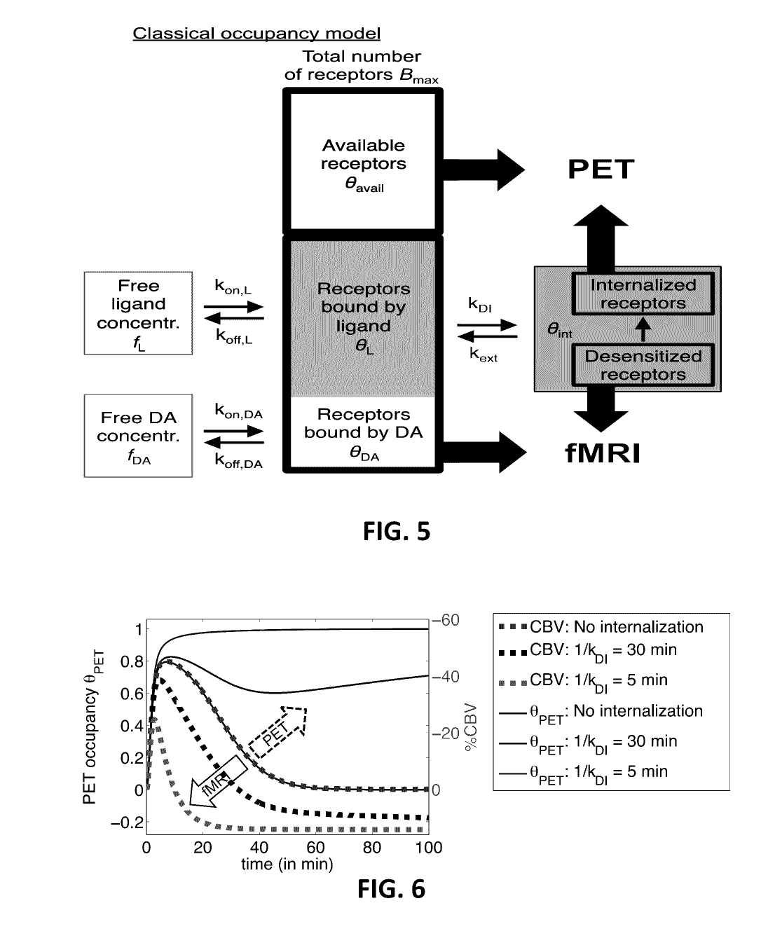

FIG. 5 illustrates the components of the RDI model and their relationship towards the classical occupancy model. High-affinity agonists can cause receptor internalization (29, 30). The process of receptor desensitization and internalization can be considered in a dynamic occupancy model. In an example of a dynamic occupancy model, the following are assumed:
i. The externalization constant kₑₓₜ is very long, and is thus negligible for timescales <2 h.
ii. Once internalized, receptors can be bound with an affinity α by the PET radiotracer. If α=0, internalized receptors do not bind the radiotracer; if 0<α<1, the affinity is reduced compared to receptors on the postsynaptic membrane and if α>1, the affinity is increased (e.g. due to the radiotracer being entrapped inside the cell membrane).
iii. Desensitized and internalized receptors do not contribute to the functional response (CBV).
iv. The rate to convert desensitized to internalized receptors is negligible with respect to the relevant experimental timescales.

The corresponding equations of this dynamic occupancy model can be expressed as follows:

$$\begin{matrix}( Eq . 5 ) & \\ \frac{d {θ}_{L}}{d t} = {k}_{off , L} ( {θ}_{avail} {f}_{L} ( \frac{{K}_{D , DA}}{{K}_{D , L}} ) \frac{{θ}_{DA}^{( 0 )}}{( 1 - {θ}_{DA}^{( 0 )} )} - {θ}_{L} ) - {k}_{int} {θ}_{L} & ( a ) \\ \frac{d {θ}_{DA}}{d t} = {k}_{off , DA} ( {θ}_{avail} {f}_{DA} \frac{{θ}_{DA}^{( 0 )}}{1 - {θ}_{DA}^{( 0 )}} - {θ}_{DA} ) & ( b ) \\ \frac{d {θ}_{int}}{d t} = {k}_{DI} ( {θ}_{L} + {θ}_{DA} ) & ( c ) \\ {θ}_{avail} = 1 - {θ}_{L} - {θ}_{DA} - {θ}_{int} & ( d )\end{matrix}$$

In this set of equations, θ is the fractional occupancy as described for Error! Reference source not found. The parameters kₒₙ and k_{off} denote the association and dissociation rate constants for either ligand L or dopamine DA (as specified in the subscript). The concentrations of the free ligand and free DA can both be normalized to the baseline DA occupancy at time zero, such that f describes the fraction of free concentration (relative to baseline DA). The parameter θᵢₙₜ describes the fraction of internalized receptors and k_{DI} denotes the desensitization and internalization rate constant. The above equations, plus the measurement models for PET and fMRI are a full description of the RDI model employed in this study for simulation and parameter estimation.

The model described in Error! Reference source not found. reduces to the classical occupancy model for the case of k_{DI}=0. In this case, the model can describe a dynamic description of how antagonists and full or partial agonist occupancies are related to changes in CBV, under the assumption that no receptor adaptation mechanisms occur. To investigate the effect of drug efficacy on the CBV response, efficacies were varied while assuming an initial 20% basal DA occupancy at D2R (31).

### Example 2: Experimental and Simulation Results

Two animals (male rhesus macaques: M1 (8 years) and M2 (6 years)) underwent PET/MR imaging. For each study, the animal was anesthetized, initially with 10 mg/kg ketamine and 0.5 mg/kg xylazene, and maintained with isoflurane (1%, mixed with oxygen) after intubation.

[¹¹C]Raclopride was injected using a bolus+infusion protocol. Infusions employed k_{bol} values (32) of 52 min and 81 min for animals M1 and M2, respectively. Boluses were administered by hand over a duration of 30 seconds, after which infusion at a rate of 0.01 ml/s was started with an automatic pump (Medrad Spectra Solaris). Specific activities at time of injection were 3.7±2.8 mCi/nmol. At ˜30 minutes, the high-affinity D2/D3 agonist quinpirole (K_{D,D2}=576 nM, K_{D,D3}=5 nM (33)) was injected at one of three different doses (0.1, 0.2, 0.3 mg/kg) selected to span a range of occupancies from about 30 to 80%. Experiments and repeated administration of pharmacological challenges in all NHPs were separated by at least 2 weeks, so that results were not influenced from prior history. To test whether a single quinpirole injection desensitizes the fMRI response to subsequent injections, the quinpirole injection was repeated 2 h after the first dose in two sessions in animal M2 (0.1 and 0.2 mg/kg doses). In two separate experimental sessions, the lower affinity D2/D3 agonist ropinirole (K_{D,D2}=970 nM, K_{D,D3}=61 nM (25)) was injected at either 0.1 mg/kg or 0.3 mg/kg in animal M2. Additionally, the D2 antagonist prochlorperazine was injected at doses 0.1 and 0.2 mg/kg in animal M2 to serve as a direct comparison to the agonists, and to investigate whether the temporal relationship between occupancy and function that was reported for the short-acting antagonist raclopride (10) was maintained for an antagonist with much longer duration of action. Although the employed ligands have affinity for both D2 and D3, the results in caudate-putamen, as determined in this study, are dominated by D2 since the D2/D3 ratio in the striatum is ˜95% (46).

Simultaneous PET and MR data were acquired on a prototype scanner that includes a BrainPET insert and a Tim Trio 3T MR scanner (Siemens AG, Healthcare Sector, Erlangen Germany). A custom-built tight-fitting PET compatible 8-channel NHP receive array (34) together with a vendor-supplied local circularly polarized transmit coil was used for MR imaging.

The phased array enabled 2-fold acceleration with GRAPPA (35) in the anterior-posterior direction. Whole-brain fMRI data were acquired for the duration of PET imaging with multi-slice echo-planar imaging (EPI) that had an isotropic resolution of 1.3 mm and a temporal resolution of 3 s (TR). Other parameters included FOV_{MR}=110×72.8 mm², BW=1350 Hz/pixel, flip angle=60° and an echo time of 23 ms (TE). To improve fMRI detection power, ferumoxytol (Feraheme, AMAG Pharmaceuticals, Cambridge Mass.) was injected at 10 mg/kg prior to fMRI (36).

PET emission data were acquired in list-mode format for 100 min starting with radiotracer injection. Images were reconstructed with a 3D Poisson ordered-subset expectation maximization algorithm using prompt and variance-reduced random coincidence events. Normalization, scatter and attenuation sinograms (including attenuation of the radiofrequency coil) were included in the reconstruction (37). The reconstructed volume consisted of 1.25×1.25×1.25 mm voxels in a 256×256×153 matrix, which were downsampled by a factor of 2 post-reconstruction. Framing intervals were 10×30 sec, followed by 1 min frames.

PET and MR data were registered to the Saleem-Logothetis stereotaxic space (38) with an affine transformation (12 degrees of freedom) using a multi-subject MRI template (39), in which standard regions of interest (ROI) were defined based on anatomy. Alignment of the EPI data used an affine transformation plus local distortion fields. After motion-correcting using AFNI software (40), and after spatially smoothing fMRI data with a 2.5 mm Gaussian kernel, statistical analysis was carried out using the general linear model (GLM). The temporal response to the drug injection was modeled with a gamma-variate function, in which the time-to-peak was adjusted to minimize the χ²/DOF of the GLM fit to the data. A long-lasting signal change that was observed in most brain regions and non-specific to the striatum was modeled with a sigmoidal GLM regressor but not included in the temporal drug profile due to its non-specificity. Additionally, regressors that correspond to translations in three dimensions and were derived from motion correction were used in the GLM analysis. The resulting signal changes were then converted to percentage changes in CBV by standard methods (41).

PET kinetic modeling employed a general linear model formulation of the simplified reference tissue model (SRTM) (14), with cerebellum as the reference ROI, and the rate constant (k₂) for cerebellum derived from the high-binding region putamen using a 2-parameter SRTM model (42). Since binding does not stay constant but is dynamically altered as a result of the D2 agonist drug challenge, the kinetic analysis included the time-dependent parameter k₂ₐ(t) (43, 44) which was converted to a “dynamic binding potential” (DBP) (10).

All PET and fMRI data analysis and generation of parametric images from voxel-wise kinetic modeling were generated with open-access software (www.nitrc.org/projects/jip). In first-level fixed-effects analyses, all doses exhibited significant responses throughout basal ganglia by both PET and fMRT. As shown in FIG. 5, statistical values used for maps were computed by regularizing the random-effects variance using about 100 effective degrees-of-freedom in the mixed-effects analysis following the Worsley method (45).

The RDI model was used to generate a family of CBV curves for RDI rates in the range of 1 min to 30 min. An estimate of the RDI constant for each dataset was then achieved by minimizing the residual norm between the model and data after drug injection.

PET imaging data and fMR imaging data in anesthetized non-human primates were acquired with the radiotracer [¹¹C]raclopride. Three pharmacological agents specific to D2/D3 receptors—high-affinity agonist quinpirole, lower-affinity agonist ropinirole and antagonist prochlorperazine—at different doses were used to determine the effect of ligand occupancy, affinity, and efficacy on receptor desensitization and internalization. To test whether a single quinpirole injection desensitizes the fMRI response to subsequent injections, the quinpirole injection was repeated 2 hours after the first dose.

Parametric maps from PET kinetic modeling results (DBP_{ND}^{peak} maps) and fMRI statistical analysis (CBV^{peak} maps) from three doses of quinpirole injections in two animals each are shown in FIG. 1. All maps show the imaging data from 2 animals, analyzed with a mixed-effects model. The upper row of FIG. 1 depicts dynamic binding potential maps (DBP_{ND}^{peak}) showing specific binding of the radiotracer [¹¹C]raclopride in the striatum decreases with increasing quinpirole. The lower row of FIG. 1 depicts voxelwise maps showing % CBV^{peak} changes, windowed by a p-value map with p<0.03. As quinpirole dose increases, the negative CBV signal that is specific to the striatum increases in magnitude.

Ropinirole injections showed a similar spatial distribution. Specific binding from PET and negative CBV changes from fMRI both had a localized response in putamen and caudate only. Positive CBV changes were observed for higher doses outside of the striatum, in thalamus and vermis of the cerebellum. Moreover, the measurements from both modalities were dose-dependent. Specific binding decreased with increasing dose of quinpirole. CBV signal showed a progressively larger magnitude with increasing dose. Quantitative values from the putamen ROI are listed in Table 1 below for two animals and each dose for quinpirole and ropinirole.

TABLE 1
Summary of PET/fMRI experimental outcomes in the putamen for the D2/D3
agonist injections (quinpirole and ropinirole) in two animals (M1, M2).

| | Quinpirole | | Quinpirole | | Quinpirole | | Ropinir. | Ropinir. |
|---|---|---|---|---|---|---|---|---|
| | Dose 1 | | Dose 2 | | Dose 3 | | Dose 1 | Dose 2 |
| Injected mass (mg/kg) | 0.1 | | 0.2 | | 0.3 | | 0.1 | 0.3 |
| Putamen ROI | M1 | M2 | M1 | M2 | M1 | M2 | M1 | M1 |
| BP⁽⁰⁾ | 4.2 | 5.6 | 3.6 | 5.7 | 2.8 | 5.1 | 5.1 | 5.7 |
| DBP_{ND}^{peak} | 2.8 | 4.0 | 1.8 | 2.5 | 0.7 | 1.8 | 4.7 | 2.9 |
| Peak Occ. \hat{θ}^{peak} (%) | 33 | 29 | 50 | 56 | 74 | 64 | 7 | 49 |
| Peak CBV (%) | −2.0 | −3.9 | −5.7 | −4.2 | −5.8 | −5.0 | −6.6 | −9.7 |

FIG. 2 shows a plot of % CBV peak values against peak occupancy for three doses of quinpirole for animal M2 in the putamen and caudate ROIs, together with a power law fit. The plot compares peak CBV responses (CBV^{peak}) to peak occupancies in whole putamen and caudate from animal M2. Error bars denote the within-session uncertainty from the GLM analysis. The relationship between CBV and occupancy is described by a monotonically decreasing function and shows the dependency of the CBV and occupancy signals with dose. Caudate had a larger signal magnitude compared to caudate for all occupancy levels. Values from all quinpirole and ropinirole doses for two animals are listed in Table 1. Data points were described with a power law fit (a(θ^{peak})^{b}) to illustrate that CBV^{peak} changes exhibit a monotonically decreasing function versus the occupancy of D2/D3 agonist.

FIG. 3 shows a representative PET time activity curve (TAC) for caudate and the reference region (cerebellum), together with the kinetic modeling fit from dynamic 2-parameter simplified reference tissue model (14), for one (0.2 mg/kg) of the three doses of quinpirole injection for animal M2. The upper row of FIG. 3 shows a PET time activity curves for the caudate and cerebellum ROIs for the 0.2 mg/kg quinpirole injection at 35 min (for animal M2), with kinetic modeling fits from 2-parameter SRTM with cerebellum as the reference (black line). The arrow at 35 min indicates the time at which the quinpirole challenge was administered. The simultaneously acquired % CBV timecourse is shown in the bottom row. In particular, the lower row of FIG. 3 shows corresponding CBV timecourses indicating a negative response due to the challenge in the caudate ROI. A second injection of 0.2 mg/kg quinpirole in the same session did not produce a measurable CBV response in the caudate.

For the three doses of 0.1, 0.2 and 0.3 mg/kg quinpirole injections in animals M1 and (M2), the % CBV signal change peaked at 3 (2.3), 2.3 (1.7) and 2.2 (2.1) minutes, and returned to baseline within 15.1 (16.1), 8.5 (12.1) and 15.6 (14.5) minutes. Hence, for all doses, the % CBV signal change peaked within a few minutes and returned to baseline relatively quickly. The duration of the CBV signal was defined as starting with the onset of the gamma-variate GLM regressor until it returned to <0.1% absolute CBV signal change. An additional sigmoidal GLM regressor that modeled a slow rise of CBV signal to above baseline was not included in the temporal profile as it was non-specific to the striatum and observable in most brain regions. Contrary to the very fast striatal CBV response, the PET signal decreased and remained low for the duration of the experiment in all cases. Additionally, kinetic modeling of the PET TACs showed that changes in dynamic binding potential DBP(t) were better described by a sigmoidal function (according to the Akaike criterion) than any gamma-variate function.

A second quinpirole injection (for the 0.1 and 0.2 mg/kg doses) 2 hours after the first dose was administered to test if the first response could be replicated or had been altered. As shown in FIG. 3, in both experimental sessions with the two doses, the second injection showed no detectable change in the fMRI signal, consistent with expectations of receptor internalization.

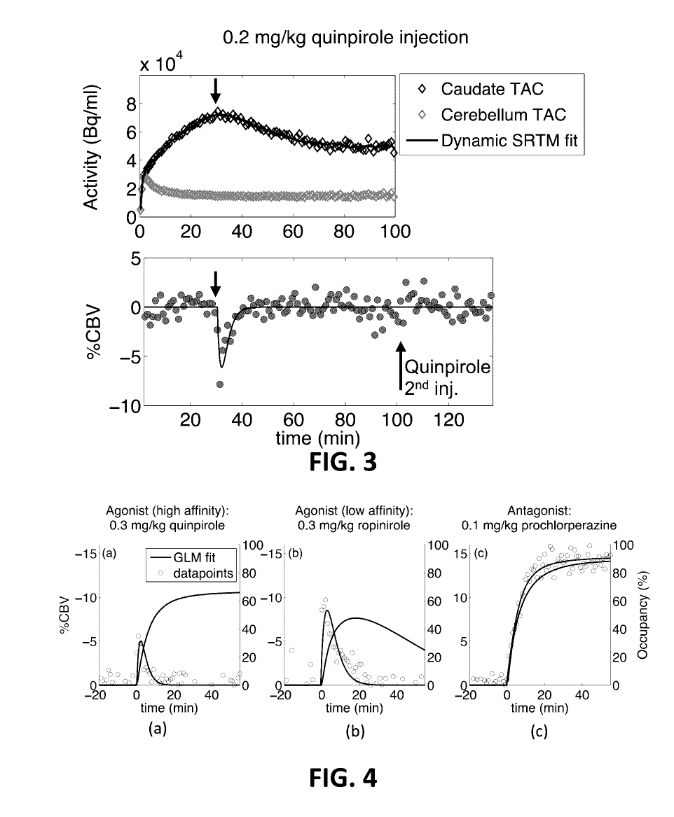

FIG. 4 shows timecourses of CBV (green, red) and occupancy (blue) resulting from exposure to three different pharmacological challenges in animal M2. Graph (a) of FIG. 4 shows that the high affinity agonist quinpirole elicited a very short CBV response, whereas PET occupancy stays elevated for the duration of the experiment. Graph (b) of FIG. 4 shows that the agonist ropinirole had a lower affinity compared to quinpirole and resulted in a slightly longer, though still short, CBV response, whereas occupancy peaked at 17.8 min and then started to decrease. Compared to quinpirole, ropinirole displayed a larger CBV^{peak} signal at lower occupancy. Graph (c) of FIG. 4 shows that the antagonist prochlorperazine indicated that CBV and occupancy timecourses were matched, demonstrating that CBV could stay elevated for longer durations in time. The antagonist showed a reversed CBV sign and the largest magnitude compared to the agonists. Overall, the discrepancy in time between CBV and occupancy and diminished CBV magnitude for the agonists suggests that RDI affects both PET and fMRI imaging data, and can vary with drug affinity and potency.

The temporal response from a 0.1 and 0.3 mg/kg ropinirole injection showed robust negative CBV responses localized to the striatum. As shown in FIG. 4, compared to quinpirole, the CBV timecourse was similar in shape but peaked slightly later, at 4.0 and 3.4 min after the injection for each dose. The return to baseline was also slower, with the CBV response in caudate due to the 0.1 mg/kg ropinirole dose lasting 28.5 min and that due to the 0.3 mg/kg dose lasting 25.0 min. However, the PET response for ropinirole showed that occupancy did not stay elevated but peaked at 17.8 min after which it decreased. In agreement with this, kinetic modeling showed that DBP(t) was better described with a gamma-variate rather than sigmoidal function. The fMRI and PET timecourses for ropinirole were thus less divergent than for quinpirole. In addition, the CBV^{peak} magnitude from quinpirole was smaller despite its higher potency compared to ropinirole, consistent with predictions of the RDI model. Quinpirole showed a CBV^{peak} signal of −5%, whereas the less potent agonist ropinirole displayed a CBV^{peak} signal of −8.5%, despite reaching a lower occupancy.

Prochlorperazine administration was employed as a control since, as a D2/D3 antagonist, no desensitization or internalization is expected. Graph (c) of FIG. 4 shows that the fits to the CBV timecourse and occupancy measures matched each other, suggesting that both timecourses represent the dynamics of the injected drug itself at the postsynaptic membrane. Compared to the agonists, the CBV response from prochlorperazine resulted in the largest peak magnitude of 14.4% at 88% occupancy. These results are in concordance with the classical occupancy theory that does not include RDI.

By fitting a dynamic occupancy model to experimental data, the in vivo desensitization and internalization time constants were estimated. 1/k_{DI} was estimated to be 5±1 min (mean±std) for the 3 doses of the D2/D3 agonist quinpirole. Estimates of 1/k_{DI} for the less potent D2/D3 agonist ropinirole were 8.5±2.1 min.

Simulation results from the dynamic occupancy model based on the classical theory were developed. In addition simulations were extended to incorporate RDI as an additional feature. The simulation results were investigated to predict varying ligand properties. FIG. 5 illustrates an entire compartmental model and its relationship to PET and fMRI signals. In particular, FIG. 5 shows a schematic illustrating the compartmental model that describes receptor desensitization and internalization at dopaminergic synapses. The total number of receptors (Bₘₐₓ) is composed of available receptors at the postsynaptic membrane, those bound by an injected agonist, those bound by endogenous dopamine and desensitized/internalized receptors. Occupied receptors are in exchange with free ligand in the synaptic space. Receptors that are occupied by agonist trigger desensitization and internalization. Since externalization mechanisms are known to be very slow, kₑₓₜ is assumed to be zero for the duration of timecourses modeled. The parameters that determine the PET and fMRI signal changes are depicted in blue and green, respectively. This shows that PET and fMRI timecourses contain complementary information about receptor adaptation mechanisms.

Receptor desensitization and internalization's effect on the functional (hemodynamic) response as measured by changes in cerebral blood volume (CBV), and PET temporal profiles through simulations of a dynamic occupancy model (Eq. 5) was measured. CBV responses were modeled with a neurovascular coupling model from Eq. 2 and occupancies were computed using Eq. 4. Unless specified otherwise, a full agonist is assumed to have an efficacy of 1, and a radiotracer is assumed not to bind to internalized receptors (α=0).

FIG. 6 shows the simulation results for three different rates of RDI. In particular, the simulation results from the model of receptor desensitization and internalization show how PET and fMRI signal timecourses are affected for different rates of RDI (k_{DI}) due to a D2/D3 agonist injection at time t=0. Without RDI (red curves, k_{DI}=0), CBV follows the timecourse of receptor occupancy by drug. If no RDI occurs, PET and fMRI signals are matched in time.

With very short time constants (5 min), the fMRI timecourse is shortened, whereas PET occupancy stays elevated for much longer. If RDI occurs with a moderate time constant of 30 min, PET and fMRI signals start to diverge. If agonist-induced RDI occurs with time constants on the order of 90 min or less (30 and 5 min are shown as an example), the PET and CBV temporal responses diverge noticeably on the timescale of a typical [¹¹C]-PET experiment. The fMRI signal duration is shortened and PET occupancy stays elevated for a longer time when compared to drug occupancy in the absence of RDI. The temporal divergence increases as internalization time constants become shorter. Without RDI, the CBV timecourse peaks at 7 min and returns to baseline at 56 min, consistent with the occupancy of drug. A fast RDI constant of 5 min (green curves) causes the CBV peak to be shifted to 2.8 min and fall below baseline at 10 min. In addition, the magnitude of the CBV signal decreases as RDI constants become shorter. Peak occupancies of 80% would be expected to evoke a peak CBV signal of −45% without RDI, based upon prior antagonist studies and given the assumption that basal DA occupancy is about 20%. Simulated peak CBV amplitudes decrease to −39% and −25% with RDI time constants of 30 and 5 min, respectively. Shorter RDI time constants reduce CBV magnitude at all occupancy levels in simulations.

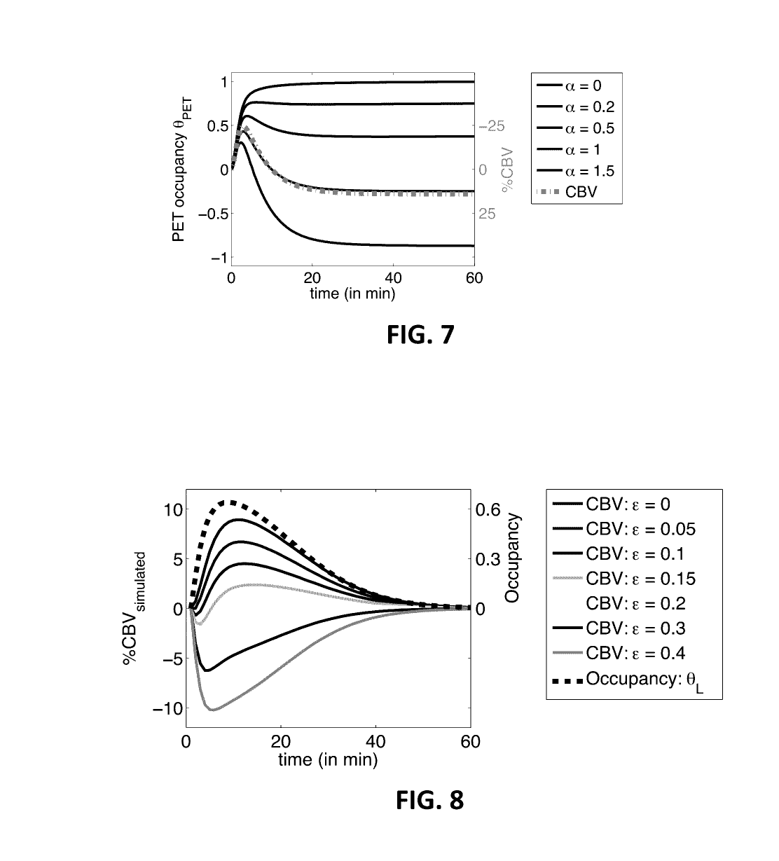

FIG. 7 depicts simulated PET occupancy measures for the duration of a dynamic PET scan for different affinities of internalized receptors relative to external receptors (α), assuming an RDI time constant of 5 min. FIG. 7 shows how the prolonged decrease in the PET signal, which corresponds to a long-lasting increase in receptor occupancy, depends not only on the RDI constant but also on the affinity of the radiotracer for internalized receptors.

Fast RDI time constants (e.g. 5 min) cause a larger percentage of receptors to be internalized, thus resulting in higher peak occupancy levels compared to a slow internalization constant. If internalized receptors are not accessible to the radiotracer (α=0), occupancy is very high and stays elevated for the duration of the experiment. If the internalized receptors are low-affinity receptors (0<α<1), occupancy is still increased for a prolonged time. Occupancy remains elevated (i.e., binding potential stays suppressed) for a prolonged time.

If affinity for internalized receptors does not change or is increased (α>1), occupancy can decrease to negative values, indicating that the time activity curve would show an increase due to the agonist exposure. If the radiotracer can bind with equal affinity to internalized receptors (α=1), the timecourse of occupancy matches that of CBV. If affinity for the radiotracer is increased due to internalization (α=1.5), the simulations show that binding potential will be increased from its baseline value and thus will result in a negative occupancy, producing a paradoxical response.

FIG. 8 shows simulated CBV timecourses within the classical occupancy model (without RDI) for the same occupancies but varying efficacies of a ligand. Simulations of the classical occupancy model (Eq. 5, with kDI=0) with the coupling model from Eq. 2, predict CBV temporal responses for a range of theoretical ligands with varying efficacies. Baseline occupancies of endogenous dopamine are assumed to be 20%. Antagonists (ε=0) show a positive CBV signal, whereas agonists (ε>1) show a negative CBV signal. For partial agonists, the response depends on the basal DA occupancy: If efficacy is high enough, the partial agonist response is similar to a full agonist response. If efficacy is low, the CBV response of a low-efficacy partial agonist (0<ε≦0.2) becomes biphasic.

CBV responses from all ligands in the context of the classical model, i.e., assuming no desensitization or internalization, approximately follow the shape of the occupancy timecourse. For a ligand with efficacy zero (i.e. an antagonist), the CBV response is purely positive, whereas for full agonists or partial agonists with efficacy larger than basal occupancy, the CBV response is purely negative. Both cases exhibit a similar timecourse except for the sign of the response, which largely conformed to the steady-state prediction of Eq. 3: (i) for efficacies larger than the basal occupancy of the endogenous neurotransmitter, the CBV response is similar to that of a full agonist, but with diminished magnitude; (ii) for efficacies significantly smaller than the basal occupancy of the endogenous neurotransmitter, the CBV response is similar to an antagonist. An interesting temporal response occurred at efficacies just below the basal occupancy (i.e., for a weak partial agonist). In this case, the simulated response is biphasic, albeit with a small overall magnitude.

Selective D2/D3 agonists can elicit dose-dependent increases in receptor occupancy, together with decreases in CBV, in the striatum of anesthetized non-human primates. These spatial and dose correlations between changes in D2/D3 receptor occupancy and changes in CBV support a neurovascular coupling mechanism during receptor-specific activation. This relationship holds for D2/D3 agonists, as shown in this study with quinpirole and ropinirole, as well as for D2/D3 antagonists, including prochlorperazine and raclopride (10), and can be described by a neurovascular coupling model (Eq. 2). Consistent with the known coupling of D2/D3 GPCR stimulation, negative CBV changes in the striatum, an in vivo measurement of receptor-specific functional inhibition, were observed. These results conform to positive CBV changes being observed due to antagonism at D2/D3, as previously reported (10). Agonist-induced CBV changes observed outside of the striatum at high doses could indicate activation of regions interconnected to the striatum or secondary effects of drug exposure.

Dynamic PET and fMRI signals after exposure to D2/D3 agonists (quinpirole and ropinirole) exhibited a pronounced temporal dissociation: While receptor binding of [¹¹C]raclopride stayed decreased for a prolonged time, CBV signals returned to baseline rapidly. Compared to the reported binding offset time of ˜20 minutes for the agonist quinpirole (15), CBV responses were much shorter. In contrast, decreases in occupancy measured with [¹¹C]raclopride persisted much longer. As shown in FIG. 4, since PET and fMRI temporal responses were observed to be matched for D2/D3 antagonists (10), this temporal divergence in the case of agonists suggests additional physiological mechanisms that modulate signals in the case of agonist exposure.

Several PET studies have suggested that a persistent decrease in PET signal is due to agonist-induced receptor internalization (1, 13, 16). An artificially prolonged increase in occupancy can occur in wildtype mice after amphetamine exposure but not in mice that have a knockout of the arrestin-3, or β-arrestin-2, gene (ARRB2)—a crucial link for the internalization of receptors (2). Suppression of the PET signal was still evident after 4 hours in wildtype animals, whereas the signal had returned to baseline levels in ARRB2-knockout mice. This suggests that once receptors are internalized, this state may be preserved for several hours, with receptors being either recycled or degraded afterwards (17). The PET data using an exogenous agonist concur with this interpretation since a prolonged decrease in the availability of D2/D3 receptors was observed, together with the fact that receptor function did not recover with a second injection of the D2/D3 agonist quinpirole after 2 hours.

The absence of an fMRI response with a second injection of the D2/D3 agonist quinpirole after 1-2 h is in agreement with electrophysiological recordings. These show that reapplication of quinpirole fails to initiate a second response in dopaminergic neurons of the ventral tegmental area (18). Although receptor signals can be transduced through the β-arrestin pathway (47), the functional consequences mainly include endocytosis and ERK activation. The magnitude and timing is expected to be much less compared to the amplification of the G-protein-coupled pathway. A second response due to a second quinpirole injection is not expected. Moreover, D2Rs may undergo degradation post endocytosis (18), which can further contribute to the long recovery period of D2 receptor availability. The latter result is in agreement with electrophysiological recordings, in which reapplication of quinpirole fails to initiate a second response in dopaminergic neurons of the ventral tegmental area (18). D2 receptors may undergo degradation post-endocytosis (18), which may further contribute to the long recovery period of D2 receptor availability.

The D2/D3 agonist quinpirole has been shown to induce receptor internalization rapidly in vitro (1). As an initial step of this process, receptors desensitize due to phosphorylation, which decouples them from G protein signaling and causes them to become functionally inactive. Due to this disconnect in the functional signaling chain, fMRI signal was expected to subside as receptors desensitized. This effect was expected to abbreviate the fMRI signal even when drug was available for binding. All doses of quinpirole induced fMRI responses that reached peak magnitude within minutes and lasted less than 30 min, suggesting that receptor desensitization and internalization occurs rapidly.

To look at effects that may alter RDI rates, the D2/D3 agonist ropinirole was used. Ropinirole is about seven times less potent compared to quinpirole (19). A lower-affinity agonist was hypothesized to reduce the rate at which receptors desensitize. The CBV results show a slower return to baseline for ropinirole, suggesting that receptor desensitization is not as pronounced compared to the more potent agonist quinpirole.

Receptor desensitization and internalization are closely linked mechanisms that allow dynamic regulation of cell signaling. Initial desensitization after agonist exposure occurs by phosphorylation of receptors through kinases (20), which causes receptors to become functionally inactive. This desensitization mechanism would itself cause the fMRI signal to abbreviate, but could neither explain the prolonged binding decrease nor the lack of a 2^{nd} response after several hours since dephosporylation occurs on the same timescale of minutes (21). Rather, it has been shown that phosphorylation can be succeeded by recruitment of β-arrestin-2, thus triggering receptor internalization after initial desensitization (22). Given the reported time frames of these mechanisms, fMRI may initially reflect desensitization, whereas PET may be more sensitive to long-term binding properties, including internalization that lasts for hours, or even receptor degradation.

Alternative explanations to the RDI interpretation include the following. In in vitro settings, it has been shown that the D2R can exist in a high- and low-affinity state and can change between states in response to pharmacological injections, which may explain the observations from the conducted experiments. The state of the receptor is determined by the affinity of DA to bind, but exogenous drugs seem to have identical affinity for high- and low-affinity receptors. Although the existence of the two affinity states has been detected in homogenized tissues, in vivo states are less well established. Moreover, the high-affinity state can be functionally active, whereas the low affinity state is linked to receptor internalization. The effects on both PET and fMRI data may thus be the result of a change in affinity states, providing a way to image different affinity states of the D2/D3 receptor.

The time course of the observed CBV signal on its own could possibly be explained without desensitization if quinpirole is a weak partial agonist, which should exhibit a biphasic response within a classical occupancy model. This explanation, in some cases, may not simultaneously account for the PET response, which would need to resolve towards baseline much more rapidly, as shown in FIG. 4. Alternatively, the data could be explained by desensitization without internalization if quinpirole binds to synaptic D2/D3 receptors for the duration of the experiment. However, quinpirole binding to the receptor is not expected to last several hours, with 1/k_{off} being ˜20 minutes (15). Additionally, there is evidence from in vitro studies that quinpirole is an agonist causing receptor internalization with high functional potency (23), and is often used a reference for evaluating the efficacy of other D2/D3 agonists (24, 25). Receptor desensitization, followed by internalization, may be the mechanism underlying the observations of divergence between apparent occupancy and function in vivo.

The examples of the dynamic occupancy model described herein suggest how receptor desensitization and internalization can produce measurable signal changes using concurrent PET and fMRI. If receptors desensitize and internalize rapidly, the dynamic occupancy models can predict that PET and fMRI signals are driven in opposite directions from the expected binding and response profile of the drug, as shown in FIG. 6. The PET and fMRI data are consistent with this the dynamic occupancy models of receptor internalization. The temporal divergence can thus serve as a quantifiable measure of internalization rate, with more rapid desensitization and internalization producing a greater divergence. Conversely, antagonist-induced responses are consistent with a classical occupancy model that does not require invocation of a desensitization mechanism.

A number of features within the model of RDI described herein were investigated to address the signal mechanisms underlying these non-invasive imaging modalities. In particular, the effect of affinity changes of internalized receptors to available ligands was investigated. While neurotransmitters like dopamine do not cross cell membranes, PET ligands appear to access internalized receptors with altered affinity. Some data suggest that raclopride cannot access internalized receptors (16), while others report binding with a reduced affinity (1). If the affinity of internalized receptors does not change, then the PET signal is a measure of total receptor availability, although a subset of these receptors would be functionally inactive. If the affinity to internalized receptors is reduced, then a component of observed elevated occupancy (Error! Reference source not found.) reflects a low affinity rather than increased binding density. Conversely, if internalization increases the affinity for the radioligand by preventing efflux more than influx of the radioligand, then PET could report drug-induced reductions in apparent receptor occupancy. This scenario has been hypothesized as an explanation for paradoxical results obtained using spiperone (11).

The RDI model also predicts that peak magnitude of the CBV response decreases with shorter RDI time constants, which is consistent with the experimental data. Although quinpirole is known to be more potent than ropinirole, it produced a smaller CBV magnitude at higher occupancy, for example, as shown in FIG. 4. This can be explained by a shorter internalization time constant for quinpirole, which is consistent with the rate estimates. The RDI rate-dependent magnitude changes can thus modify the CBV versus occupancy relationship, which may provide an additional indication (apart from temporal dissociations) for differentiating RDI rates among agonists.

The methods of dynamic simultaneous PET/fMRI measurements described herein combined with the appropriate multimodal signal model of RDI enable a measurement of receptor desensitization and internalization in vivo and a quantitative estimate of rates. Comparing results from the model to the experimental data, the time constants for RDI using quinpirole are about 5 min. This is consistent with in vitro measurements of internalization rate constants, which also are reported to be on the order of 5 min (1). The applications of this work can be used to compare compounds well-characterized in vitro to serve as a basis for interpreting in vivo dynamics. Integrating complementary information from both PET and fMRI enables observation of in vivo desensitization mechanisms non-invasively and explore the nature of functional receptor dynamics.

Measuring RDI dynamics in vivo can be clinically important as receptor states and dynamics may be affected in disease and further modified through drug therapy. Full or partial D2 agonists are sometimes approved for treatment, e.g., of movement disorders or psychiatric disease. The methods described herein can be used to determine the effects of short and long-term exposure of drugs on receptor dynamic function. The dynamic function of other receptor systems can also be affected by RDI, which can be characterized using the methods described herein. For example, the regulation of receptor trafficking in, for example, the glutamate system has been linked to schizophrenia (26) and altered serotonin and dopamine levels have been suggested to affect 5-HT_{2A} internalization (27), thus playing an important role for drug treatments in anxiety and depression. The ability to measure RDI dynamics in patients can be applicable to therapeutic treatment, can be used to evaluate the efficacy of drugs in a number of neurological and neuropsychiatric disease states, and can provide insight into disease mechanisms.

The effects of pharmacological doses of D2/D3 agonist injections in anesthetized non-human primates were investigated. The dynamic occupancy model can describe receptor desensitization and internalization and its effect on PET/fMRT data. Experimental data with a D2/D3 agonist showed that PET and fMRT signals match in anatomical space and with injected dose. PET and CBV timecourses diverged, with PET specific binding staying suppressed for a prolonged amount of time relative to CBV signals, which were only transient. Estimated rates of RDI from the data are in agreement with those from in vitro literature. Overall, the dynamic occupancy model can provide first measurements of receptor internalization dynamics in vivo with simultaneous PET/fMRT. Characterizing dynamic receptor adaptation mechanisms in vivo has the potential to inform drug development and evaluation, and to expand understanding of the long-term effects of drug exposure.

The experimental results and simulations illustrate the dynamics of dopamine receptor desensitization and internalization. Non-invasive, in vivo measurements of receptor adaptations can be collected. The D2/D3 agonist quinpirole, which induces receptor internalization in vitro, was administered at graded doses in a primate model while imaging with simultaneous PET and fMRI. Results showed a pronounced temporal divergence between receptor occupancy, which remained elevated following agonist infusion, and fMRI signal, which responded transiently. Experimental comparisons to an antagonist (prochlorperazine) and a lower-affinity agonist (ropinirole) suggest that the temporal dissociation between occupancy and function represents desensitization and internalization, and depends upon drug efficacy and affinity.

Measuring RDI dynamics in vivo can be used to elucidate drug responses and to establish links to behavior. Repeated exposure to agonists in rodents and NHPs has been linked to supersensitivity (49) and this phenomenon has been suggested to have a role in the development of schizophrenia (48). Behavioral patterns change versus time and dose, with biphasic patterns attributed to pre-vs postsynaptic function (50; 51) or to a balance between excitatory and inhibitory stimulation (52). In some cases, imaging data can be acquired in combination with readouts of occupancy, function, and behavioral patterns in awake animals. The behavioral patterns can be associated with a predefined value of the dynamic response of the functional state and/or the dynamic response of the receptor occupancy. In this regard, the dynamic response of the functional state and/or the dynamic response of the receptor occupancy, in some cases, are indicative of a behavioral pattern, e.g., associated with a disorder such as schizophrenia.

Receptor states and dynamics can be affected in disease and further modified through drug therapy. Full or partial D2 agonists are approved for the treatment of, e.g., movement disorders and psychiatric disease. The dynamic function of other receptor systems has also been suggested to be affected by RDI. The 5-HT2A receptor system has shown similar observations as the D2/D3 receptor system that can be attributed to internalization, though further insight into RDI would be needed. The regulation of receptor trafficking in, e.g., the glutamate system has been linked to schizophrenia (26) and altered serotonin and DA levels have been suggested to affect 5-HT2A internalization (27), thus having an important role for drug treatments in anxiety and depression. The ability to measure RDI dynamics in vivo could help clarify disease mechanisms, advance therapeutic treatment, and evaluate drug efficacy in neurological and neuropsychiatric disorders.

A number of implementations have been described. Nevertheless, it will be understood that various modifications may be made. Accordingly, other implementations are within the scope of the claims.

### REFERENCES

The following publications are incorporated by reference in their entirety and are referenced in parentheses throughout the following disclosure.
1. Guo N et al. Impact of D2 Receptor Internalization on Binding Affinity of Neuroimaging Radiotracers. Neuropsychopharmacology 2009; 35(3):806-817.
2. Skinbjerg M et al. D2 dopamine receptor internalization prolongs the decrease of radioligand binding after amphetamine: A PET study in a receptor internalization-deficient mouse model. NeuroImage 2010; 50(4):1402-1407.
3. Clark A J. General pharmacology. Berlin; New York: Springer-Verlag; 1937:
4. Maehle A-H, Prüll C-R, Halliwell RF. The emergence of the drug receptor theory. Nat. Rev. Drug Discov. 2002; 1(8):637-641.
5. Breier A et al. Schizophrenia is associated with elevated amphetamine-induced synaptic dopamine concentrations: Evidence from a novel positron emission tomography method. Proc. Natl. Acad. Sci. 1997; 94(6):2569-2574.
6. Laruelle M et al. Microdialysis and SPECT measurements of amphetamine-induced dopamine release in nonhuman primates. Synapse 1997; 25(1):1-14.
7. Chen Y C I et al. Detection of dopaminergic neurotransmitter activity using pharmacologic MRI: Correlation with PET, microdialysis, and behavioral data. Magn. Reson. Med. 1997; 38(3):389-398.
8. Chen Y I et al. Detection of dopaminergic cell loss and neural transplantation using pharmacological MRI, PET and behavioral assessment. Neuroreport 1999; 10(14):2881-2886.
9. Choi J-K, Chen Y I, Hamel E, Jenkins B G. Brain hemodynamic changes mediated by dopamine receptors: Role of the cerebral microvasculature in dopamine-mediated neurovascular coupling. NeuroImage 2006; 30(3):700-712.
10. Sander C Y et al. Neurovascular coupling to D2/D3 dopamine receptor occupancy using simultaneous PET/functional MRI. Proc. Natl. Acad. Sci. 2013; 201220512.
11. Chugani D C, Ackermann R F, Phelps M E. In Vivo [3H]Spiperone Binding: Evidence for Accumulation in Corpus Striatum by Agonist-Mediated Receptor Internalization. J. Cereb. Blood Flow Metab. 1988; 8(3):291-303.
12. Ginovart N. Imaging the Dopamine System with In Vivo [11C]raclopride Displacement Studies: Understanding the True Mechanism. Mol. Imaging Biol. 2005; 7(1):45-52.
13. Laruelle M. Imaging Synaptic Neurotransmission With in Vivo Binding Competition Techniques: A Critical Review. J. Cereb. Blood Flow Metab. 2000; 20(3):423-451.
14. Lammertsma A A, Hume S P. Simplified Reference Tissue Model for PET Receptor Studies. NeuroImage 1996; 4(3):153-158.
15. Levant B, Grigoriadis D E, DeSouza E B. Characterization of [3H]quinpirole binding to D2-like dopamine receptors in rat brain. J. Pharmacol. Exp. Ther. 1992; 262(3):929-935.
16. Sun W, Ginovart N, Ko F, Seeman P, Kapur S. In Vivo Evidence for Dopamine-Mediated Internalization of D2-Receptors after Amphetamine: Differential Findings with [3H]Raclopride versus [3H]Spiperone. Mol. Pharmacol. 2003; 63(2):456-462.
17. Beaulieu J-M, Gainetdinov R R. The Physiology, Signaling, and Pharmacology of

Dopamine Receptors. Pharmacol. Rev. 2011; 63(1):182-217.
18. Bartlett S E et al. Dopamine responsiveness is regulated by targeted sorting of D2 receptors. Proc. Natl. Acad. Sci. U.S.A 2005; 102(32):11521-11526.
19. Berger A K, Green T, Siegel S J, Nestler E J, Hammer R P. cAMP Response Element Binding Protein Phosphorylation in Nucleus Accumbens Underlies Sustained Recovery of Sensorimotor Gating Following Repeated D2-Like Receptor Agonist Treatment in Rats. Biol. Psychiatry 2011; 69(3):288-294.
20. Seeman P. Are dopamine D2 receptors out of control in psychosis?. Prog. Neuropsychopharmacol. Biol. Psychiatry 2013; 46:146-152.
21. Williams J T et al. Regulation of μ-Opioid Receptors: Desensitization, Phosphorylation, Internalization, and Tolerance. Pharmacol. Rev. 2013; 65(1):223-254.
22. Beaulieu J-M, Del' Guidice T, Sotnikova T, Lemasson M, Gainetdinov R. Beyond cAMP: the regulation of Akt and GSK3 by dopamine receptors. Front. Mol. Neurosci. 2011; 4:38.
23. Gardner B, Strange P G. Agonist action at D2 (long) dopamine receptors: ligand binding and functional assays. Br. J. Pharmacol. 1998; 124(5):978-984.
24. Coldwell M C, Boyfield I, Brown T, Hagan J J, Middlemiss D N. Comparison of the functional potencies of ropinirole and other dopamine receptor agonists at human D_{2(long)}, D₃ and D_{4.4} receptors expressed in Chinese hamster ovary cells. Br. J. Pharmacol. 1999; 127(7):1696-1702.
25. Perachon S, Schwartz J-C, Sokoloff P. Functional potencies of new antiparkinsonian drugs at recombinant human dopamine D1, D2 and D3 receptors. Eur. J Pharmacol. 1999; 366(2-3):293-300.
26. Hall J, Trent S, Thomas K L, O'Donovan M C, Owen M J. Genetic Risk for Schizophrenia: Convergence on Synaptic Pathways Involved in Plasticity [Internet]. Biol. Psychiatry doi:10.1016/j.biopsych.2014.07.011
27. Bhattacharyya S, Raote I, Bhattacharya A, Miledi R, Panicker M M. Activation, internalization, and recycling of the serotonin 2A receptor by dopamine. Proc. Natl. Acad. Sci. 2006; 103(41):15248-15253.
28. Mandeville J B et al. A receptor-based model for dopamine-induced fMRI signal. NeuroImage 2013; 75:46-57.
29. Goggi J L, Sardini A, Egerton A, Strange P G, Grasby P M. Agonist-dependent internalization of D2 receptors: Imaging quantification by confocal microscopy. Synapse 2007; 61(4):231-241.
30. Lane D A et al. Quinpirole elicits differential in vivo changes in the pre- and postsynaptic distributions of dopamine D2 receptors in mouse striatum: relation to cannabinoid-1 (CB1) receptor targeting. Psychopharmacology (Berl.) 2012; 221(1):101-113.
31. Verhoeff N P L G et al. Dopamine depletion results in increased neostriatal D(2), but not D(1), receptor binding in humans. Mol. Psychiatry 2002; 7(3):233, 322-328.
32. Carson R E et al. Comparison of Bolus and Infusion Methods for Receptor Quantitation: Application to [18F]Cyclofoxy and Positron Emission Tomography. J Cereb Blood Flow Metab 1993; 13(1):24-42.
33. Sokoloff P, Giros B, Martres M-P, Bouthenet M-L, Schwartz J-C. Molecular cloning and characterization of a novel dopamine receptor (D3) as a target for neuroleptics. Nature 1990; 347(6289):146-151.
34. Sander C Y et al. Low 511 keV-Attenuation Array Coil Setup for Simultaneous PET/MR Imaging of the Monkey Brain. In: Proc. Intl. Soc. Mag. Reson. Med. 21. Salt Lake City, Utah: 2013:
35. Griswold M A et al. Generalized autocalibrating partially parallel acquisitions (GRAPPA). Magn. Reson. Med. 2002; 47(6):1202-1210.
36. Mandeville J B. IRON fMRI measurements of CBV and implications for BOLD signal. NeuroImage 2012; 62(2):1000-1008.
37. Catana C et al. Toward Implementing an MRI-Based PET Attenuation-Correction Method for Neurologic Studies on the MR-PET Brain Prototype. J Nucl. Med. 2010; 51(9):1431-1438.
38. Saleem K S, Logothetis N K. A Combined MRI and Histology Atlas of the Rhesus Monkey Brain. [Internet]. Burlington: Elsevier; 2006:
39. McLaren D G et al. A population-average MRI-based atlas collection of the rhesus macaque. Neuroimage 2009; 45(1):52-59.
40. Cox R W. AFNI: What a long strange trip it's been. NeuroImage 2012; 62(2):743-747.
41. Mandeville J B et al. Dynamic functional imaging of relative cerebral blood volume during rat forepaw stimulation. Magn. Reson. Med. 1998; 39(4):615-624.
42. Ichise M et al. Linearized Reference Tissue Parametric Imaging Methods: Application to [11C]DASB Positron Emission Tomography Studies of the Serotonin Transporter in Human Brain. J Cereb. Blood Flow Metab. 2003; 23(9):1096-1112.
43. Normandin M D, Schiffer W K, Morris E D. A linear model for estimation of neurotransmitter response profiles from dynamic PET data. NeuroImage 2012; 59(3):2689-2699.
44. Alpert N M, Badgaiyan R D, Livni E, Fischman A J. A novel method for noninvasive detection of neuromodulatory changes in specific neurotransmitter systems. NeuroImage 2003; 19(3):1049-1060.
45. Worsley K J et al. A General Statistical Analysis for fMRI Data. NeuroImage 2002; 15(1):1-15.
46. Cumming P (2011). Absolute abundances and affinity states of dopamine receptors in mammalian brain: A review. Synapse 65: 892-909.
47. Lefkowitz R J, Shenoy S K (2005). Transduction of Receptor Signals by β-Arrestins. Science 308: 512-517.
48. Seeman M V, Seeman P (2014). Is schizophrenia a dopamine supersensitivity psychotic reaction? Prog Neuropsychopharmacol Biol Psychiatry 48: 155-160.
49. Bolaños-Jiménez Rodrigo, Arizmendi-Vargas Jorge, Martinez-Zavala Néstor, Carrillo-Ruiz José Damián, Calderón Álvarez-Tostado José Luis, Jiménez-Ponce Fiacro, et al (2011). Quinpirole Effects on the Dopaminergic System. Br J Pharmacol Toxicol 2: 310.
50. Arnsten A F, Cai J X, Steere J C, Goldman-Rakic P S (1995). Dopamine D2 receptor mechanisms contribute to age-related cognitive decline: the effects of quinpirole on memory and motor performance in monkeys. J Neurosci 15: 3429-3439.
51. Eilam D, Szechtman H (1989). Biphasic effect of D-2 agonist quinpirole on locomotion and movements. Eur J Pharmacol 161: 151-157.
52. Winstanley C A, Zeeb F D, Bedard A, Fu K, Lai B, Steele C, et at (2010). Dopaminergic modulation of the orbitofrontal cortex affects attention, motivation and impulsive responding in rats performing the five-choice serial reaction time task. Behav Brain Res 210: 263-272.

What is claimed is:

1. A method comprising:
administering to a subject (i) a pharmacological agent that binds to receptors in a subject, and (ii) a radiotracer to alter a functional state and occupancy of the receptors in the subject;
acquiring imaging data of brain tissue in the subject comprising the receptors, the imaging data comprising positron emission tomography (PET) imaging data and functional magnetic resonance (fMR) imaging data; and
calculating (i) a dynamic response of the functional state to the pharmacological agent and the radiotracer based on the fMR imaging data, and (ii) a dynamic response of the receptor occupancy to the pharmacological agent and the radiotracer based on the PET imaging data.

2. The method of claim 1, wherein the pharmacological agent and the radiotracer are administered to the subject substantially simultaneously, within 5-10 minutes of each other, or within 2-3 hours of each other.

3. The method of claim 1, further comprising administering an iron oxide contrast agent before acquiring the imaging data.

4. The method of claim 1, wherein the pharmacological agent or the radiotracer is administered to the subject parenterally.

5. The method of claim 1, wherein the receptor occupancy corresponds to receptor occupancy of the pharmacological agent on the receptors.

6. The method of claim 1, wherein the radiotracer is a ligand for the receptors.

7. The method of claim 1, wherein acquiring the imaging data comprises simultaneously acquiring the PET imaging data and the fMR imaging data.

8. The method of claim 1, wherein acquiring the imaging data comprises sequentially acquiring the PET imaging data and the fMR imaging data.

9. The method of claim 1, wherein the imaging data include images representing a brain of the subject.

10. The method of claim 1, wherein the dynamic response of the functional state is defined at least in part by a peak value of the functional state or a temporal response of the functional state, and the dynamic response of the receptor occupancy is defined at least in part by a peak value of the receptor occupancy or a temporal response of the receptor occupancy.

11. The method of claim 1, wherein calculating the dynamic response of the functional state comprises calculating the dynamic response of the functional state based on a hemodynamic response of the subject.

12. The method of claim 11, further comprising calculating the hemodynamic response based on a cerebral blood volume of the subject measured from the imaging data.

13. The method of claim 1, wherein calculating the dynamic response of the receptor occupancy comprises calculating the dynamic response of the receptor occupancy based on basal receptor occupancy.

14. The method of claim 1, wherein calculating the dynamic response of the receptor occupancy comprises calculating the dynamic response of the receptor occupancy based on a binding potential of the receptors.

15. The method of claim 1, further comprising quantifying receptor trafficking of the subject based on the dynamic response of the functional state and the dynamic response of the receptor occupancy.

16. The method of claim 15, wherein quantifying receptor trafficking comprises computing at least one of a desensitization rate constant, an internalization rate constant, a change in affinity of the receptors, or a change in efficacy of the pharmacological agent.

17. The method of claim 1, further comprising determining specificity, efficacy, affinity, or neurovascular coupling parameters of the radiotracer or the pharmacological agent.

18. The method of claim 1, further comprising classifying the radiotracer or the pharmacological agent based on the dynamic response of the functional state and the dynamic response of the receptor occupancy.

19. The method of claim 18, wherein classifying the radiotracer or the pharmacological agent comprises classifying the radiotracer or the pharmacological agent as a classification selected from the group consisting of antagonist, inverse agonist, partial agonist, and full agonist.

20. The method of claim 1, further comprising measuring a neurological effect of the pharmacological agent based on the receptor occupancy.

21. The method of claim 20, wherein measuring the neurological effect comprises measuring occupancy peak values or response duration after administering the pharmacological agent.

22. One or more computer-readable non-transitory media storing instructions that are executable by a processing device, and upon execution cause the processing device to perform operations comprising:
receiving imaging data of a subject representing receptors of the subject after a pharmacological agent and a radiotracer are administered to the subject, the imaging data comprising PET imaging data and fMR imaging data; and
calculating (i) a dynamic response, to the pharmacological agent and the radiotracer, of a functional state based on the fMR imaging data, and (ii) a dynamic response, to the pharmacological agent and the radiotracer, of a receptor occupancy based on the PET imaging data.

23. A system comprising:
a computing device comprising
a memory configured to store instructions; and
a processor to execute the instructions to perform operations comprising
receiving imaging data of a subject representing receptors of the subject after a pharmacological agent and a radiotracer are administered to the subject, the imaging data comprising PET imaging data and fMR imaging data; and
calculating (i) a dynamic response, to the pharmacological agent and the radiotracer, of a functional state based on the fMR imaging data, and (ii) a dynamic response, to the pharmacological agent and the radiotracer, of a receptor occupancy based on the PET imaging data.

24. The system of claim 23, further comprising:
an fMR imaging device to acquire the fMR imaging data representing the receptors, and
an PET imaging device to acquire the PET imaging data representing the receptors.
